SEQUENCE LISTING (1 sequence)

SEQ ID NO: 1 - protein, 1044 aa, Homo sapiens
MPPGVDCPMEFWTKEENQSVVVDFLLPTGVYLNFPVSRNANLSTIKQLLWHRAQYEPLFHMLSGPEAYVFTCINQTAEQQELEDEQRRLCDVQPFLPVLRLVAREGDRVKKLINSQISLLIGKGLHEFDSLCDPEVNDFRAKMCQFCEEAAARRQQLGWEAWLQYSFPLQLEPSAQTWGPGTLRLPNRALLVNVKFEGSEESFTFQVSTKDVPLALMACALRKKATVFRQPLVEQPEDYTLQVNGRHEYLYGSYPLCQFQYICSCLHSGLTPHLTMVHSSSILAMRDEQSNPAPQVQKPRAKPPPIPAKKPSSVSLWSLEQPFRIELIQGSKVNADERMKLVVQAGLFHGNEMLCKTVSSSEVSVCSEPVWKQRLEFDINICDLPRMARLCFALYAVIEKAKKARSTKKKSKKADCPIAWANLMLFDYKDQLKTGERCLYMWPSVPDEKGELLNPTGTVRSNPNTDSAAALLICLPEVAPHPVYYPALEKILELGRHSECVHVTEEEQLQLREILERRGSGELYEHEKDLVWKLRHEVQEHFPEALARLLLVTKWNKHEDVAQMLYLLCSWPELPVLSALELLDFSFPDCHVGSFAIKSLRKLTDDELFQYLLQLVQVLKYESYLDCELTKFLLDRALANRKIGHFLFWHLRSEMHVPSVALRFGLILEAYCRGSTHHMKVLMKQGEALSKLKALNDFVKLSSQKTPKPQTKELMHLCMRQEAYLEALSHLQSPLDPSTLLAEVCVEQCTFMDSKMKPLWIMYSNEEAGSGGSVGIIFKNGDDLRQDMLTLQMIQLMDVLWKQEGLDLRMTPYGCLPTGDRTGLIEVVLRSDTIANIQLNKSNMAATAAFNKDALLNWLKSKNPGEALDRAIEEFTLSCAGYCVATYVLGIGDRHSDNIMIRESGQLFHIDFGHFLGNFKTKFGINRERVPFILTYDFVHVIQQGKTNNSEKFERFRGYCERAYTILRRHGLLFLHLFALMRAAGLPELSCSKDIQYLKDSLALGKTEEEALKHFRVKFNEALRESWKTKVNWLAHNVSKDNRQ

(19) United States
(12) Patent Application Publication
(10) Pub. No.: US 2013/0116267 A1
(43) Pub. Date: May 9, 2013

(54) Role of PI3K P110 Delta Signaling in Retroviral Infection and Replication

(71) Applicants: Peter D. Katsikis, Merion Station, PA (US); Alina C. Boesteanu, Willow Grove, PA (US); Martin Turner, Balsham (GB)

(72) Inventors: Peter D. Katsikis, Merion Station, PA (US); Alina C. Boesteanu, Willow Grove, PA (US); Martin Turner, Balsham (GB)

(21) Appl. No.: 13/809,713

(22) PCT Filed: Jul. 13, 2011
(86) PCT No.: PCT/US11/43881

§ 371 Date: Jan. 11, 2013

Related U.S. Application Data
(63) Continuation of application No. 12/835,474, filed on Jul. 13, 2010.

(51) Int. Cl. A61K 31/52; A61K 31/506; A61K 31/519

(52) U.S. Cl. 514/263.21; 435/375

(57) ABSTRACT

The invention includes compositions and methods for regulating PI3K p110 delta as an anti-retroviral therapy. The invention includes a method of inhibiting p110 delta, a component of PI3K p110 delta signaling pathway, or any combination thereof in a cell as an anti-retroviral therapeutic approach for treating a retroviral infection, for example HTV. The invention includes a method of modulating PI3K p110 delta in a cell infected with a retrovirus by contacting the cell with an effective amount of a composition comprising an inhibitor of PI3K P110 delta.

### CROSS-REFERENCE TO RELATED APPLICATION

This application claims priority to U.S. application Ser. No. 12/835,474, filed Jul. 13, 2010, which application is hereby incorporated herein by reference in its entirety.

### BACKGROUND OF THE INVENTION

A retrovirus is an RNA virus that is replicated in a host cell via the enzyme reverse transcriptase to produce DNA from its RNA genome. The DNA is then incorporated into the host's genome by an integrase enzyme. The virus thereafter replicates as part of the host cell's DNA, Retroviruses are enveloped viruses that belong to the viral family Retroviridae.

The retrovirus itself stores its nucleic acid in the form of a +mRNA (including the 5′cap and 3′PolyA inside the virion) genome and serves as a means of delivery of that genome into cells it targets as an obligate parasite, and constitutes the infection. Once in the host's cell, the RNA strands undergo reverse transcription in the cytosol and are Integrated into the host's genome, at which point the retroviral DNA is referred to as a provirus. Retroviruses are difficult to detect the virus until they have infected the host.

Simply, the retrovirus enters a host cell and provokes the RNA strands Inside of the normally-functioning cell to undergo reverse transcription. Normally, DNA would be transcribed into RNA, and RNA would translate into proteins. However, when a retrovirus is inside of a cell, the first two steps of that process would be switched. (Rather than DNA→RNA→Protein, it would be RNA->DNA) The host cell would become a provirus as this has occurred.

Human immunodeficiency virus (HIV) is a lentivirus (a member of the retrovirus family) that causes acquired immunodeficiency syndrome (AIDS), a condition in humans in which the immune system begins to fail, leading to life-threatening opportunistic infections, Infection with HIV occurs by the transfer of blood, semen, vaginal fluid, pre-ejaculate, or breast milk. Within these bodily fluids, HIV is present as both free virus particles and virus within Infected immune cells. The four major routes of transmission are unsafe sex, contaminated needles, breast milk, and transmission from an infected mother to her baby at birth (vertical transmission).

HIV infection in humans is considered pandemic by the World Health Organization (WHO). From its discovery in 1981 to 2006, AIDS killed more than 25 million people. HIV infects about 0.6% of the world's population. In 2005 alone, AIDS claimed an estimated 2.4-3.3 million lives, of which more than 570,000 were children. A third of these deaths are occurring in Sub-Saharan Africa, retarding economic growth and increasing poverty. According to current estimates, HIV is set to infect 90 million people in Africa, resulting in a minimum estimate of 18 million orphans. Antiretroviral treatment reduces both the mortality and the morbidity of HIV infection, but routine access to antiretroviral medication is not available in all countries.

HIV infects primarily vital cells in the human immune system such as helper T cells (to be specific, CD4+ T cells), macrophages, and dendritic cells. HIV infection leads to low levels of CD4+ T cells through three main mechanisms: direct viral killing of infected cells; increased rates of apoptosis in infected cells; and killing of infected CD4+ T cells by CD8 cytotoxic lymphocytes that recognize infected cells. When CD4+ T cell numbers decline below a critical level, cell-mediated immunity is lost, and the body becomes progressively more susceptible to opportunistic infections.

Most untreated people infected with HIV eventually develop AIDS. The majority of these individuals die from opportunistic infections or malignancies associated with the progressive failure of the immune system. HIV progresses to AIDS at a variable rate affected by viral, host, and environmental factors. Most individuals will progress to AIDS within 10 years of HIV infection. Treatment with anti-retrovirals increases the life expectancy of people infected with HIV. Even after HIV has progressed to diagnosable AIDS, the average survival time with antiretroviral therapy was estimated to be more than 5 years as of 2005. Without antiretroviral therapy, someone who has AIDS typically dies within a year.

PI3K represent a family of enzymes that phosphorylate D-myo-phosphatidylinositol (PtdIns) or its derivatives on the 3-hydroxyl of the inositol group (Vanhaesebroeck et al., 2001, Annu. Rev. Biochem. 70:535-602). PI3Ks are classified as class I, II, or III, depending on their subunit structure, regulation, and substrate selectivity (Vanhaesebroeck et al., 2001, Annu. Rev. Biochem. 70:535-602; Fruman et al., 2002, Semin. Immunol. 14:7-18). PI3K belonging to class I are heterodimers composed of a catalytic subunit of approximately 110 kDa, and a tightly associated regulatory subunit that modulates the activity and cellular location of the enzyme. Four isoforms (p110α, p111β, p110γ, and p110δ) of the catalytic subunits of class I PI3K exist (Vanhaesebroeck et al., 2001, Annu. Rev. Biochem. 70:535-602; Fruman et al., 2002, Semin. Immunol. 14:7-18). PI3K p110δ (or pt 10 delta; protein sequence SEQ ID NO: 1) is expressed preferentially by hematopoietic cells (Vanhaesebroeck et al., 1997, Proc. Natl. Acad. Sci. USA 94:4330-4335; Chantry et al., 1997, J. Biol. Chem. 272:19236-19241) and plays an important role in B and T cell development and function (Okkenhaug et al., 2003, Nat. Rev. Immunol. 3:317-330; Okkenhaug et al., 2002, Science 297:1031-1034; Clayton et al., 2002, J. Exp. Med. 196:753-763; Okkenhaug et al., 2006, J. Immunol. 177:5122-5128).

Current drugs that reduce or block HIV infection or replication in a subject have significant side effects in the subject, such as drug-related toxicity, lipodystrophy, dyslipidemia, insulin resistance, increase in cardiovascular risks, and birth defects, and may lead to poor compliance by the subject. There is therefore a need in the art for drugs that may reduce or block HIV infection or replication in a subject via a novel mechanism. The present invention satisfies this need.

### BRIEF SUMMARY OF THE INVENTION

The invention includes a pharmaceutically acceptable composition for inhibiting infection by a retrovirus in a mammal. The composition comprises a pharmaceutically acceptable carrier and an inhibitor of phosphoinositide 3 kinase (PI3K) isoform p110 delta (SEQ ID NO:1), wherein the inhibitor interferes with activation of said PI3K p110 delta and replication of the retrovirus.

In one embodiment, the inhibitor interferes with pathogenesis of the retrovirus. In another embodiment, the retrovirus is HIV. In yet another embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide, a small molecule compound, and combinations thereof.

In one embodiment, the small molecule compound is selected from the group consisting of wortmannin, INK1197, KAR4000, theophylline, CAL-101, CAL-263, Compound (I) (2-[(6-amino-9H-purin-9-yl)methyl]-5-methyl-3-(o-tolyl)quinazolin-4(3H) one), Compound (II) (2-([(9H-purin-6-yl)thio]methyl)-5-chloro-3-(2-methoxyphenyl)quinazolin-4(3H)-one), Compound (II) (2-((4-amino-3-(3-hydroxyphenyl)-1H-pyrazolo[3,4-d]pyrimidin-1-yl)methyl)-5-methyl-3-(o-tolyl)quinazolin-4(3H)-one), Compound (IV) (2-(1-(6-amino-9H-purin-9-yl)ethyl)-3-phenylthieno[3,2-d]pyrimidin-4(3H)-one), Compound (V) (6-(1-((9H-purin-6-yl)thio)ethyl)-3-methyl-5-phenylisoxazolo[5,4-d]pyrimidin-4(5H)-one), Compound (VI) (3-isobutyl-6-methyl-4-oxo-N-(pyridin-3-ylmethyl)-3,4-dihydrofuro[2,3-d]pyrimidine-5-carboxamide), Compound (VII) ((S)-5-chloro-N4-(1-(8-chloro-2-(pyridin-2-yl)quinolin-3-yl)ethyl)pyrimidine-2,4-diamine), Compound (VIII) (4-(2-(1H-indazol-4-yl)-6-((4-(methylsulfonyl)piperazin-1-yl)methyl)thieno[3,2-d]pyrimidin-4-yl)morpholine), Compound (IX) (4-(6-((4-(azetidin-1-yl)piperidin-1-yl)methyl)-2-(6-fluoro-1H-indol-4-yl)thieno[3,2-d]pyrimidin-4-yl)morpholine), Compound (X) (1-((9-ethyl-2-(1H-indol-4-yl)-6-morpholino-9H-purin-8-yl)methyl)-N,N-dimethylpiperidin-4-amine), Compound (XI) (4-(2-(1H-indol-4-yl)-6-((4-methylpiperazin-1-yl)methyl)quinazolin-4-yl)morpholine), Compound (XII) (4-(2-((hexahydropyrrolo[3,4-c]pyrrol-2(1H)-yl)methyl)-5-(1H-indol-4-yl)thiazolo[4,5-d]pyrimidin-7-yl)morpholine), Compound (XIII) (N-((4-(6-fluoro-1H-indol-4-yl)-6-morpholinopyrimidin-2-yl)methyl)nicotinamide), Compound (XIV) (6-(6-fluoro-1H-indol-4-yl)-2-morpholino-N-(2-(pyridin-3-yl)ethyl)pyrimidin-4-amine), Compound (XV) (N-(2-(dimethylamino)ethyl)-4-(6-fluoro-1H-indol-4-yl)-6-morpholinopyrimidine-2-carboxamide), Compound (XVI) (5,5-dimethyl-2-morpholino-5,6-dihydrobenzo[d]thiazol-7(4H)-one), Compound (XVII) ((S)-2-(3-((1H-indol-3-yl)methyl)morpholino)-7,7-dimethyl-5,6,7,8-tetrahydro-4H-thiazolo[5,4-c]azepin-4-one), Compound (XVIII) (6,6-dimethyl-2-(6-phenyl-2H-benzo[b][1,4]oxazin-4(3H)-yl)-6,7-dihydrothiazolo[5,4-c]pyridin-4(5H)-one), Compound (XIX) (5,5-dimethyl-2-(6-phenyl-2H-benzo[b][1,4]oxazin-4(3H)-yl)-5,6-dihydrobenzo[d]-thiazol-7(4H)-one), Compound (XX) (6,6-dimethyl-2-(6-(1-methyl-1H-pyrazol-4-yl)-2H-benzo[b][1,4]oxazin-4(3H)-yl)-6,7-dihydrothiazolo[5,4-c]pyridin-4(5H)-one), Compound (XXI) (2-(6-(1-(2-hydroxy-3-methoxypropyl)-1H-pyrazol-4-yl)-2H-benzo[b][1,4]oxazin-4(3H)-yl)-6,6-dimethyl-6,7-dihydrothiazolo[5,4-c]pyridin-4(5H)-one), Compound (XXII) (6,6-dimethyl-2-(6-(6-methylpyridazin-3-yl)-2H-benzo[b][1,4]oxazin-4(3H)-yl)-6,7-dihydrothiazolo[5,4-c]pyridin-4(5H)-one), Compound (XXIII) (ethyl 2-morpholino-7-oxo-4,5,6,7-tetrahydrobenzo[b]thiophene-3-carboxylate), Compound (XXIV) ((S)-2-(3-((1H-indol-3-yl)methyl)morpholino)-4-methylthiazole-5-carboxamide), Compound (XXV) (2-(6-bromo-2H-benzo[b][1,4]oxazin-4(3H)-yl)-5,5-dimethyl-5,6-dihydrobenzo[d]thiazol-7(4H)-one), Compound (XXVI) ((S)-methyl-3-((4-(6,6-dimethyl-4-oxo-4,5,6,7-tetrahydrothiazolo-[5,4-c]pyridin-2-yl)morpholin-3-yl)methyl)-1-methyl-1H-indole-5-carboxylate), Compound (XXVII) (3,3′-(2,4-diaminopteridine-6,7-diyl)diphenol), Compound (XXVIII) (3-(2,4-diaminopteridin-6-yl)phenol), Compound (XXIX) (6-(1H-indol-4-yl)pteridine-2,4-diamine), Compound (XXX), and a pharmaceutically acceptable salt thereof, wherein in Compound (XXX) R¹ is N or CH, and R² is a substituent selected from the group consisting of 2,2-(5-thiazolidinyl-2,4-dione)-1-ethylenyl; phenyl; pyridin-2-yl; 1H-indaz-5-yl; 1H-pyrazolo[3,4-b]pyridin-5-yl; 2-amino-3-sulfonamido-pyridin-5-yl; 2-amino-3-[(N-2,4-difluorophenyl)sulfonamido]-pyridin-5-yl; 3-[(N-2,4-difluorophenyl)sulfonamide]-pyridin-5-yl; 3-(2,4-difluorobenzenosulfonylamino)-pyridin-5-yl; and 2-methoxy-3-(2,4-difluorobenzenosulfonylamino)-pyridin-5-yl.

In one embodiment, in Compound (XXX) R¹ is CH and R² is 2,2-(5-thiazolidinyl-2,4-dione)-1-ethylenyl, and the compound useful within the methods of the invention is Compound (XXXI), or a pharmaceutically acceptable salt thereof. In another embodiment, in Compound (XXX) R¹ is N and R² is 2-methoxy-3-(2,4-difluorobenzenosulfonylamino)-pyridin-5-yl, and the compound useful within the methods of the invention is Compound (XXXII), or a pharmaceutically acceptable salt thereof.

In one embodiment, the composition further comprises at least one anti-HIV drug. In another embodiment, the at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, and protease inhibitors, and combinations thereof.

In one embodiment, the composition further comprises at least one immunomodulator.

The invention also includes a method of inhibiting replication of a retrovirus in a mammalian cell. The method comprises contacting the cell with a pharmaceutically acceptable composition comprising a therapeutically effective amount of an inhibitor of PI3K p110 delta, wherein the contacting inhibits PI3K110 delta in the cell, thereby inhibiting replication of the retrovirus in the cell.

In one embodiment, the retrovirus is HIV. In another embodiment, the inhibitor is selected from the group consisting of a small interfering RNA (siRNA), a microRNA, an antisense nucleic acid, a ribozyme, an expression vector encoding a transdominant negative mutant, an antibody, a peptide, a small molecule compound, and combinations thereof.

In one embodiment, the composition further comprises at least one anti-HIV drug. In another embodiment, the at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, protease inhibitors, and combinations thereof.

In one embodiment, the composition further comprises at least one immunomodulator.

The invention also includes a method of inhibiting pathogenesis of a retrovirus in a mammalian cell. The method comprises contacting the cell with a pharmaceutically acceptable composition comprising a therapeutically effective amount of an inhibitor of PI3K p110 delta. The contacting inhibits PI3K p110 delta in the cell, thereby inhibiting pathogenesis of the retrovirus in the mammalian cell.

In one embodiment, the retrovirus is HIV. In another embodiment, the inhibitor is selected from the group consisting of a small interfering RNA (siRNA), a microRNA, an antisense nucleic acid, a ribozyme, an expression vector encoding a transdominant negative mutant, an antibody, a peptide, a small molecule, and combinations thereof.

In one embodiment, the composition further comprises at least one anti-HIV drug. In another embodiment, the at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, protease inhibitors, and combinations thereof.

In one embodiment, the composition further comprises at least one immunomodulator.

The invention also includes a method of treating or preventing infection by a retrovirus in a mammal in need thereof. The method comprises administering a pharmaceutically acceptable composition comprising a therapeutically effective amount of an inhibitor of phosphoinositide 3 kinase (PI3K) isoform p110 delta to the mammal, wherein the inhibitor interferes with activation of PI3K p110 delta and replication of the retrovirus in the mammal, thereby treating or preventing the infection in the mammal.

In one embodiment, the retrovirus is HIV. In another embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule compound.

In one embodiment, the composition further comprises at least one anti-HIV drug. In another embodiment, the at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, and protease inhibitors.

In one embodiment, the composition further comprises at least one immunomodulator.

In one embodiment, the mammal is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1C, is a series of images depicting that p110δ is expressed by lung epithelial cells and is required for influenza virus replication. FIG. 1A depicts a Western blot analysis showing the expression of p110δPI3K in the human lung epithelial cell line A549. Western blot was performed on cell lysates from A549 human lung epithelial cells, C57Bl/6 mouse splenocytes (positive control) and p110δ−/− mouse splenocytes (negative control). Asterisk indicates non-specific band. FIG. 1B is an image demonstrating that blocking p110δ PI3K activity with the specific inhibitor IC87114 (100 μM) inhibits influenza virus replication in A549 human lung epithelial cells infected with influenza virus strain PR8 (MOI=0.01). FIG. 1C is an image depicting that lung influenza virus viral load was determined in the lungs of p110δ−/− and C57Bl/6 mice infected with influenza virus strain PR8 by RT-PCR and standardized according to a viral stock of known concentration (each symbol represents one animal and horizontal lines represent median values). Viral replication was quantitated by specific RT-PCR.

FIG. 2, comprising FIGS. 2A through 2E, is a series of images demonstrating reduced morbidity and inflammation in p110δ−/− mice infected with influenza virus. p110δ−/− (white circles) and C57Bl/6 control mice (black circles) were infected with a sublethal dose of influenza virus A strain PR8. FIG. 2A is a chart depicting weight loss as measured throughout the infection until mice started to recover (means of n=11-14 animals per group shown, vertical lines represent SE; *p=0.001, **p=0.01). FIG. 2B is a chart depicting percentage of inflamed lung, Briefly, lung lobes were collected at days 6 and 10 after infection and processed for H&E staining and evaluated for tissue infiltration with immune cell (each symbol represents one mouse, horizontal lines represent means). FIG. 2C is a chart depicting the number of cells infiltrating the lungs of infected mice at 6 days post-infection as determined by using flow cytometry (each symbol represents one mouse, horizontal lines represent mean values). FIG. 2D is a chart depicting total number of NP₍₃₆₆₋₃₇₄₎-specific CD8+ T cells in the lungs of infected C57Bl/6 and p110δ−/− mice at the peak of the response (day 10) as determined by using flow cytometry and tetramers (each symbol represents one mouse, horizontal lines represent means values). FIG. 2E is a chart depicting TNFα; MCP-1, IFNγ, and MIP-2 mRNA present in the lung tissue of p110δ−/− and C57Bl/6 mice at day 6 post-infection as determined by using RT-PCR. The fold induction was calculated relative to uninfected control mice (each symbol represents one mouse, horizontal lines represent mean values).

FIG. 3, comprising FIGS. 3A and 3B, is a series of images demonstrating that inhibition of p110δ protects from lethal influenza virus infection. FIG. 3A is a chart demonstrating that p110δ−/− mice are protected from lethal challenge with a virulent influenza virus strain. p110δ−/− (solid line) and C57Bl/6 (control, dotted line) were infected with 10×LD₅₀ of virulent in mice H7N7 A/Equine/London/1416/73 influenza virus strain. FIG. 3B is a chart demonstrating that pharmacological inhibition of p110δ protects mice lethally challenged with virulent influenza virus. Wild type mice were infected with 10×LD₅₀ of virulent in mice H7N7 A/Equine/London/1416/73 influenza virus strain and were either treated with p110δ specific inhibitor IC87114 (solid line) or left treated with vehicle only (dotted line). Statistical significance is indicated in the figures.

FIG. 4 is a plot that exemplifies the reduction of HIV-1 p24 levels achieved by the PI3K p110 delta kinase inhibitor IC87114.

FIG. 5 is a series of graphs illustrating CD25 expression levels on CD4+ T cells exposed to HIV-1 in the presence or absence of the PI3K p110 delta kinase inhibitor IC87114.

FIG. 6 is a bar graph illustrating the CD25 expression levels in PHA-activated CD4+ T cells exposed to HIV-1 and the PI3K p110 delta kinase inhibitor IC87114.

FIG. 7 is a graph illustrating cell death as measured by phosphatidyl serine expression on PHA-activated CD4+ T cells exposed to HIV-1 and the PI3K p110 delta kinase inhibitor IC87114.

FIG. 8 is a bar graph illustrating the frequency of apoptotic CD4+ T cells following exposure to HIV-1 and/or the PI3K p110 delta kinase inhibitor IC87114.

### DETAILED DESCRIPTION OF THE INVENTION

The invention includes compositions and methods for regulating PI3K p110 delta kinase in a cell thereby providing a means for reducing or inhibiting retrovirus infection or replication in the cell. In one embodiment, the invention includes an inhibitor of the PI3K p110 delta kinase. In another embodiment, the inhibitor is a small molecule. In yet another embodiment, the retrovirus is HIV. Based on the disclosure presented herein, a skilled artisan would appreciate that interfering with PI3K p110 delta and downstream PI3K p110 delta signaling is useful as an anti-retroviral therapy. The methods of the invention are contemplated for use in a mammal, preferably, a human.

In one embodiment, inhibiting PI3K p110 delta may reduce retrovirus loads in infected mammals compared to the level of viral loads in an otherwise identical infected mammal where PI3K p110 delta has not been inhibited.

In another embodiment, inhibiting PI3K p110 delta may reduce level of retroviral infection of cells of mammals compared to the level of retroviral infection of cells of otherwise identical infected mammals where PI3K p110 delta has not been inhibited.

### DEFINITIONS

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or ±10%, more preferably +5%, even more preferably ±1%, and still more preferably +0.1% from the specified value, as such variations are appropriate to perform the disclosed methods.

A “subject” or “patient,” as used therein, may be a human or non-human mammal. Non-human mammals include, for example, livestock and pets, such as ovine, bovine, porcine, canine, feline and murine mammals. Preferably, the subject is human.

The term “virus” as used herein is defined as a particle consisting of nucleic acid (RNA or DNA) enclosed in a protein coat, with or without an outer lipid envelope, which is capable of replicating within a whole cell.

As used herein, the term “HIV” or “human immunodeficiency virus” refers to a member of the genus Lentivirus, part of the family of Retroviridae. The term HIV may include any strain of HIV, such as HIV-1 or HIV-2, which is capable of causing disease in a human or non-human mammal.

As used herein “endogenous” refers to any material from or produced inside an organism, cell, tissue or system. As used herein, the term “exogenous” refers to any material introduced from or produced outside an organism, cell, tissue or system.

As used herein, the term “modulate” is meant to refer to any change in biological state, i.e. increasing, decreasing, and the like. For example, the term “modulate” refers to the ability to regulate positively or negatively the expression, stability or activity of p110 delta, including but not limited to transcription of PI3K p110 delta mRNA, stability of PI3K p110 delta mRNA, translation of PI3K p110 delta mRNA, stability of PI3K p110 delta polypeptide, PI3K p110 delta post-translational modifications, PI3K p110 delta activity, or any combination thereof. Further, the term modulate may be used to refer to an increase, decrease, masking, altering, overriding or restoring of activity, including but not limited to, PI3K p110 delta activity.

As used herein, the term “inhibit” is meant to refer to a decrease change in biological state. For example, the term “inhibit” refers to the ability to regulate negatively the expression, stability or activity of p110 delta, including but not limited to transcription of PI3K p110 delta mRNA, stability of PI3K p110 delta mRNA, translation of PI3K p110 delta mRNA, stability of PI3K p110 delta polypeptide, PI3K p110 delta post-translational modifications, PI3K p110 delta activity, PI3K p110 delta signaling pathway or any combination thereof.

As used herein, the term “an inhibitor of p110 delta”, “an inhibitor of PI3K delta” or “an inhibitor of PI3Kδ” refers to any compound or molecule that detectably inhibits p110 delta.

A “PI3K p110 delta antagonist” is a composition of matter which, when administered to a mammal such as a human, detectably inhibits a biological activity attributable to the level or presence of p110 delta.

An “amino acid” as used herein is meant to include both natural and synthetic amino acids, and both D and L amino acids. “Standard amino acid” means any of the twenty L-amino acids commonly found in naturally occurring peptides. “Non-standard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides, and particularly at the carboxy- or amino-terminus, may be modified by methylation, amidation, acetylation or substitution with other chemical groups that may change a peptide's circulating half life without adversely affecting activity of the peptide. Additionally, a disulfide linkage may be present or absent in the peptides.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “fragment,” as applied to a protein or peptide, refers to a subsequence of a larger protein or peptide. A “fragment” of a protein or peptide may be at least about 20 amino acids in length; for example at least about 50 amino acids in length; at least about 100 amino acids in length, at least about 200 amino acids in length, at least about 300 amino acids in length, and at least about 400 amino acids in length (and any integer value in between).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which may be hydrolyzed into the monomeric “nucleotides.” The monomeric nucleotides may be hydrolyzed into nucleosides. As used herein polynucleotides include, but are not limited to, all nucleic acid sequences that are obtained by any means available in the art, including, without limitation, recombinant means, i.e., the cloning of nucleic acid sequences from a recombinant library or a cell genome, using ordinary cloning technology and PCR™, and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the term “fragment,” as applied to a nucleic acid, refers to a subsequence of a larger nucleic acid. A “fragment” of a nucleic acid may be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

“Isolated” means altered or removed from the natural state. For example, a nucleic acid or a peptide naturally present in a living animal is not “isolated,” but the same nucleic acid or peptide partially or completely separated from the co-existing materials of its natural state is “isolated.” An isolated nucleic acid or protein may exist in substantially purified form, or may exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences that are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Antisense” refers particularly to the nucleic acid sequence of the non-coding strand of a double stranded DNA molecule encoding a polypeptide, or to a sequence which is substantially homologous to the non-coding strand. As defined herein, an antisense sequence is complementary to the sequence of a double stranded DNA molecule encoding a polypeptide. It is not necessary that the antisense sequence be complementary solely to the coding portion of the coding strand of the DNA molecule. The antisense sequence may be complementary to regulatory sequences specified on the coding strand of a DNA molecule encoding a polypeptide, which regulatory sequences control expression of the coding sequences.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule that are matched with an anti-codon region of a transfer RNA molecule during translation of the mRNA molecule or that encode a stop codon. The coding region may thus include nucleotide residues corresponding to amino acid residues that are not present in the mature protein encoded by the mRNA molecule (e.g., amino acid residues in a protein export signal sequence).

As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, may be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “antibody,” as used herein, refers to an immunoglobulin molecule that specifically binds with an antigen. Antibodies may be intact immunoglobulins derived from natural sources or from recombinant sources and may be immunoreactive portions of intact immunoglobulins. Antibodies are typically tetramers of immunoglobulin molecules.

The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, in: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “immunoglobulin” or “Ig” is defined as a class of proteins that function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitor-urinary tracts, IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “epitope” as used herein is defined as a small chemical molecule on an antigen that may elicit an immune response, inducing B and/or T cell responses. An antigen may have one or more epitopes. Most antigens have many epitopes; i.e., they are multivalent, in general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

The term “expression” as used herein is defined as the transcription and/or translation of a particular nucleotide sequence driven by its promoter.

A “vector” is a composition of matter that comprises an isolated nucleic acid and that may be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression may be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers may be labeled with, e.g., chromogenic, radioactive, or fluorescent moieties and used as detectable moieties.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes may be labeled with, e.g., chromogenic, radioactive, or fluorescent moieties and used as detectable moieties.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may, for example, be one that expresses the gene product in a tissue specific manner.

A “constitutive” promoter is a nucleotide sequence that, when operably linked with a polynucleotide that encodes or specifies a gene product, causes the gene product to be produced in a cell under most or all physiological conditions of the cell.

An “inducible” promoter is a nucleotide sequence that, when operably linked with a polynucleotide that encodes or specifies a gene product, causes the gene product to be produced in a cell substantially only when an inducer that corresponds to the promoter is present in the cell.

A “tissue-specific” promoter is a nucleotide sequence that, when operably linked with a polynucleotide encodes or specified by a gene, causes the gene product to be produced in a cell substantially only if the cell is a cell of the tissue type corresponding to the promoter.

The term “transfected” or “transformed” or “transduced” as used herein refers to a process by which exogenous nucleic acid is transferred or introduced into the host cell. A “transfected” or “transformed” or “transduced” cell is one that has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide may differ in amino acid sequence by one or more substitutions, additions, or deletions in any combination. A variant of a nucleic acid or peptide may be a naturally occurring such as an allelic variant, or may be a variant that is not known to occur naturally. Non-naturally occurring variants of nucleic acids and peptides may be made by mutagenesis techniques or by direct synthesis.

The term “vaccine” as used herein is defined as a material used to provoke an immune response after administration of the material to a mammal.

As used herein, “vaccination” is intended for prophylactic or therapeutic vaccination.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 5′-ATTGCC-3′ and 5′-TATGGC-3′ share 50% homology.

“Parenteral” administration of an immunogenic composition includes, e.g., subcutaneous (s.c.), intravenous (i.v.), intramuscular (i.m.), or intrasternal injection, or infusion techniques.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “composition,” “pharmaceutical composition” or “pharmaceutically acceptable composition” refers to a mixture of at least one compound or molecule useful within the invention with a pharmaceutically acceptable carrier. The pharmaceutical composition facilitates administration of the compound or molecule to a patient. Multiple techniques of administering a compound or molecule exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound or molecule useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

The term “therapeutic” as used herein means a treatment and/or prophylaxis. A therapeutic effect is obtained by suppression, remission, or eradication of a disease state.

The term “treatment” as used within the context of the present invention is meant to include therapeutic treatment as well as prophylactic, or suppressive measures for the disease or disorder. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease. This includes for instance, prevention of retroviral infection or replication.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection or replication as determined by any means suitable in the art.

As used herein, an “instructional material” includes a publication, a recording, a diagram, or any other medium of expression that may be used to communicate the usefulness of the compositions and methods of the invention. The instructional material of the kit of the invention may, for example, be affixed to a container that contains the nucleic acid, peptide, and/or composition useful with the invention or be shipped together with a container that contains the nucleic acid, peptide, and/or composition. Alternatively, the instructional material may be shipped separately from the container with the intention that the instructional material and the compound be used cooperatively by the recipient.

### Description

The invention is based on the discovery that regulating or inhibiting a phosphoinositide 3 kinase (PI3K) isoform p110 delta signaling system serves to inhibit retroviral infection. A variety of components of PI3K p110 delta and downstream signaling system may serve as targets for inhibition in order to inhibit retroviral infection. In one aspect, the present invention includes a method of inhibiting PI3K p110 delta as a therapeutic target for retroviral infection. In another aspect, the present invention includes an anti-retroviral therapy comprising inhibiting at least PI3K p110 delta signaling. In yet another aspect, the retrovirus is HIV.

Compounds Useful within the Methods of the Invention

The compounds useful within the methods of the invention may be synthesized using techniques well-known in the art of organic synthesis.

In one aspect, the compound useful within the methods of the invention is a small molecule compound selected from the group consisting of wortmannin, INK1197, KAR4000, theophylline, CAL-101, CAL-263, Compound (I), Compound (II), Compound (III), Compound (IV), Compound (V), Compound (VI), Compound (VII), Compound (VIII), Compound (IX), Compound (X), Compound (XI), Compound (XII), Compound (XIII), Compound (XIV), Compound (XV), Compound (XVI), Compound (XVII), Compound (XVIII), Compound (XIX), Compound (XX), Compound (XXI), Compound (XXII), Compound (XXIII), Compound (XXIV), Compound (XXV), Compound (XXVI), Compound (XXVII), Compound (XXVIII), Compound (XXIX), and Compound (XXX), and a pharmaceutically acceptable salt thereof, as exemplified below:

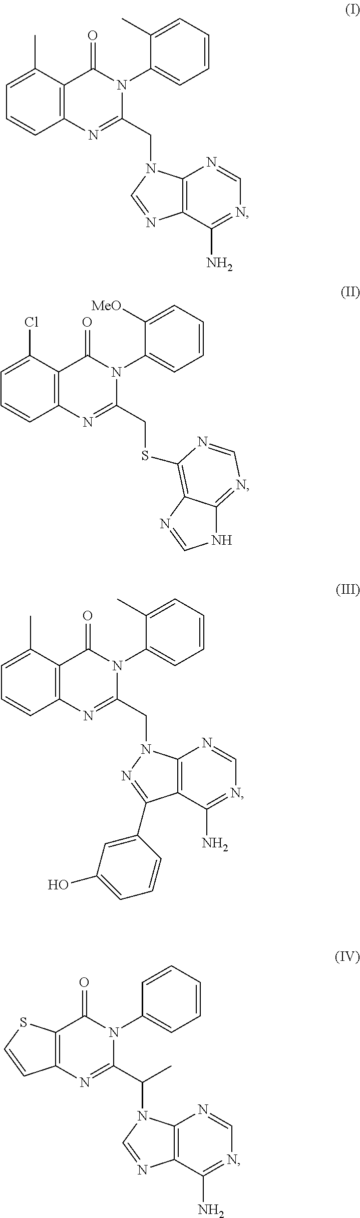 [SMILES: Cc1ccccc1-n1c(Cn2cnc3c(N)ncnc32)nc2cccc(C)c2c1=O ; COc1ccccc1-n1c(CSc2ncnc3[nH]cnc23)nc2cccc(Cl)c2c1=O ; Cc1ccccc1-n1c(Cn2nc(-c3cccc(O)c3)c3c(N)ncnc32)nc2cccc(C)c2c1=O ; CC(c1nc2ccsc2c(=O)n1-c1ccccc1)n1cnc2c(N)ncnc21]

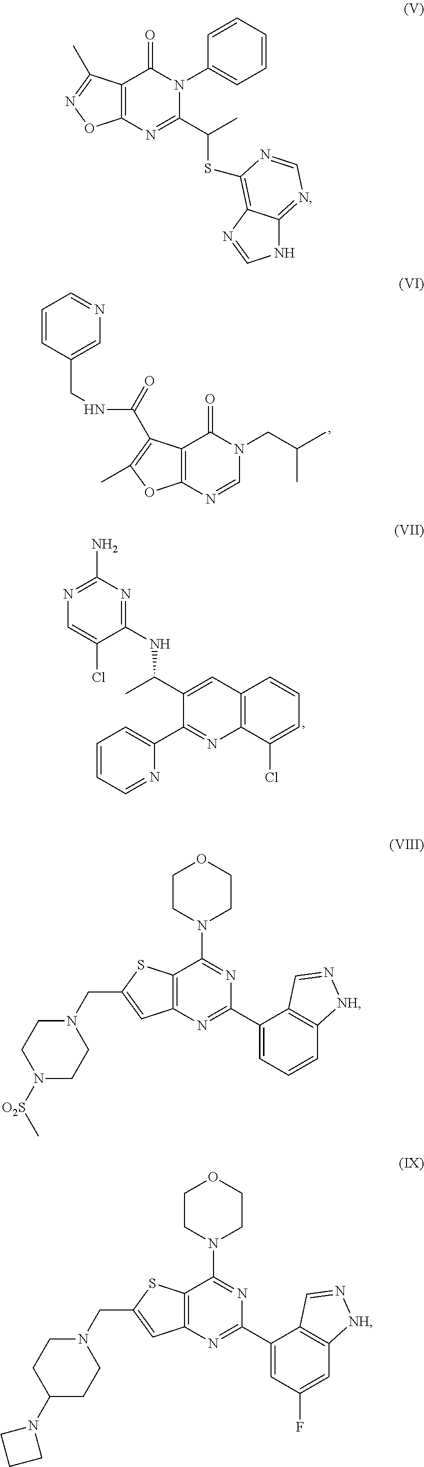 [SMILES: Cc1noc2nc(C(C)Sc3ncnc4[nH]cnc34)n(-c3ccccc3)c(=O)c12 ; Cc1oc2ncn(CC(C)C)c(=O)c2c1C(=O)NCc1cccnc1 ; C[C@H](Nc1nc(N)ncc1Cl)c1cc2cccc(Cl)c2nc1-c1ccccn1 ; CS(=O)(=O)N1CCN(Cc2cc3nc(-c4cccc5[nH]ncc45)nc(N4CCOCC4)c3s2)CC1 ; Fc1cc(-c2nc(N3CCOCC3)c3sc(CN4CCC(N5CCC5)CC4)cc3n2)c2cn[nH]c2c1]

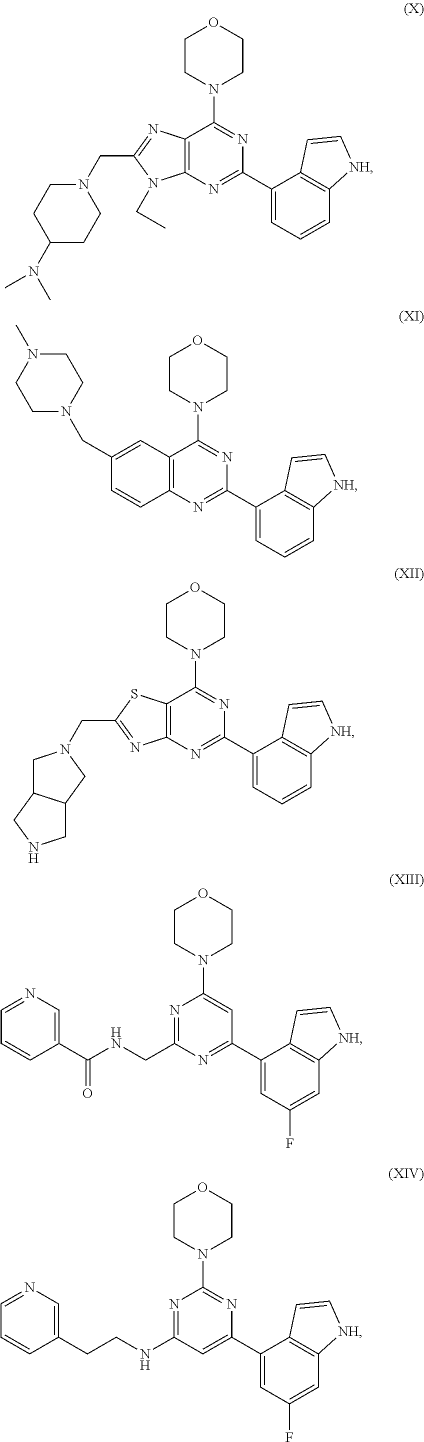

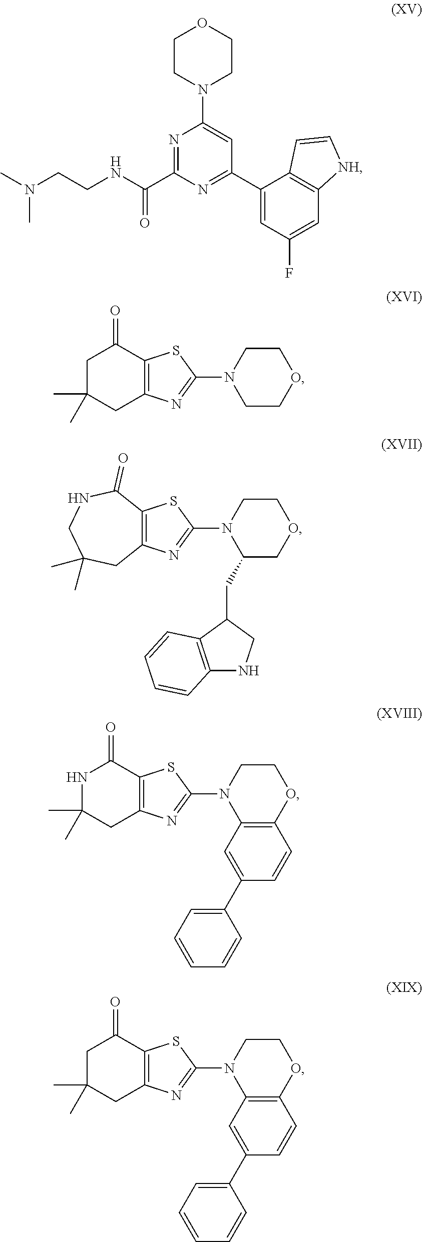 [SMILES: CN(C)CCNC(=O)c1nc(-c2cc(F)cc3[nH]ccc23)cc(N2CCOCC2)n1 ; CC1(C)CC(=O)c2sc(N3CCOCC3)nc2C1 ; CC1(C)CNC(=O)c2sc(N3CCOC[C@@H]3CC3CNc4ccccc43)nc2C1 ; CC1(C)Cc2nc(N3CCOc4ccc(-c5ccccc5)cc43)sc2C(=O)N1 ; CC1(C)CC(=O)c2sc(N3CCOc4ccc(-c5ccccc5)cc43)nc2C1]

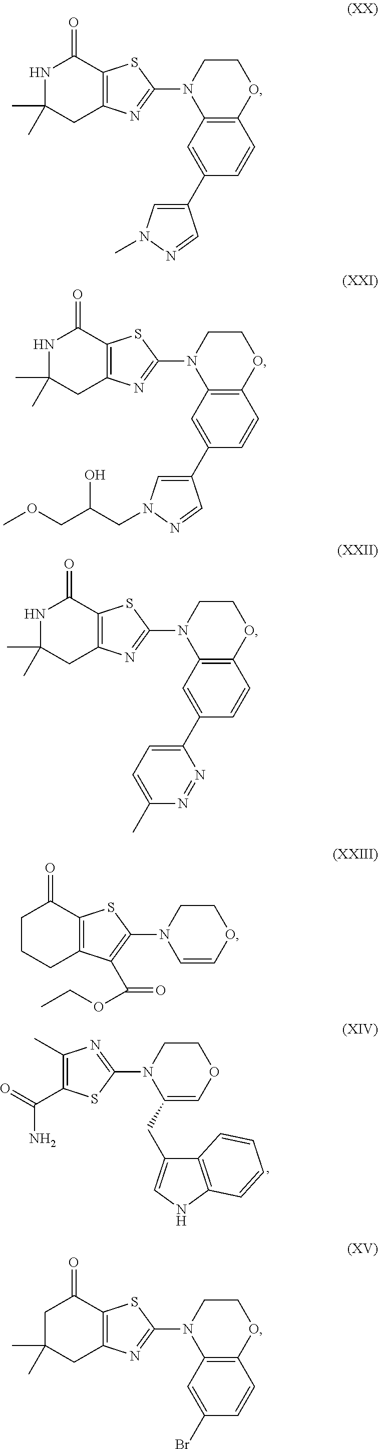

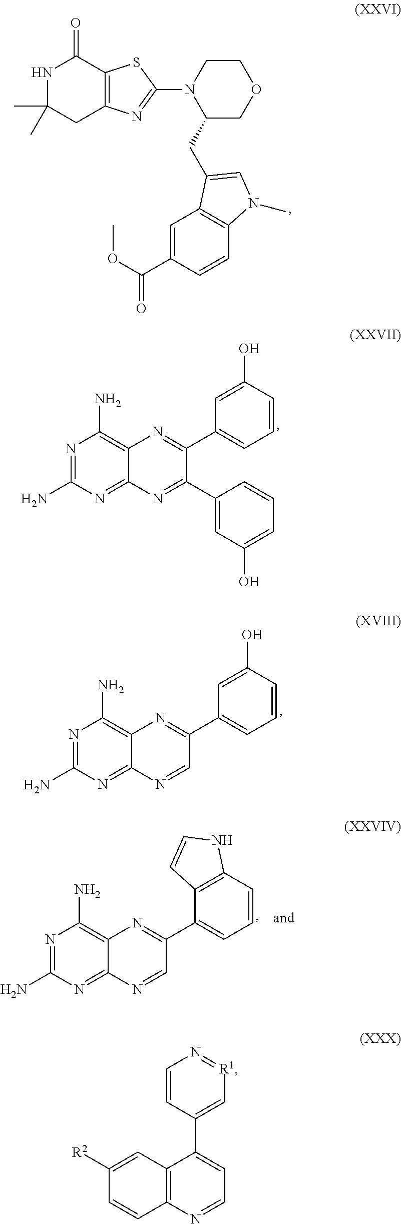

wherein in Compound (XXX) R¹ is N or CH, and R² is a substituent selected from the group consisting of 2,2-(5-thiazolidinyl-2,4-dione)-1-ethylenyl; phenyl; pyridin-2-yl; 1H-indaz-5-yl; 1H-pyrazolo[3,4-b]pyridin-5-yl; 2-amino-3-sulfonamido-pyridin-5-yl; 2-amino-3-[(N-2,4-difluorophenyl)sulfonamido]-pyridin-5-yl; 3-[(N-2,4-difluorophenyl)sulfonamide]-pyridin-5-yl; 3-(2,4-difluorobenzenosulfonylamino)-pyridin-5-yl; and 2-methoxy-3-(2,4-difluorobenzenosulfonylamino)-pyridin-5-yl; and a pharmaceutically acceptable salt thereof.

Wortmannin was reported to have an PI3K p110 delta IC₅₀ of 4.1 nM at 50 μM ATP (Ihle et al., 2005, Mol. Cancer. Ther. 4:1349-57).

Theophylline was reported to have an PI3K p110 delta IC₅₀ of 75 μM at 100 μM ATP (Foukas et al., 2002, J. Biol. Chem. 277:37124-30).

INK 1197 may be obtained from Intellikine, La Jolla, Calif.

KAR4000 may be obtained from Karus Therapeutics, Chilworth, Hampshire, UK.

CAL-101 and CAL-263 may be obtained from Calistoga Pharmaceuticals, Seattle, Wash. CAL-101 was reported to have an PI3K p110 delta of 2.5 nM (“CAL-101, An Oral p110δ Selective Phosphatidylinositol-3-Kinase (PI3K) Inhibitor for the Treatment of B Cell Malignancies Inhibits PI3K Signaling, Cellular Viability and Protective Signals of the Microenvironment”, poster, American Society of Hematology Annual meeting, Dec. 7, 2009).

Compound (I) is also known as IC87114 or 2-[(6-amino-9H-purin-9-yl)methyl]-5-methyl-3-(o-tolyl)quinazolin-4(3H)-one. Compound (I) or a pharmaceutically acceptable salt thereof may be prepared using methods known to those skilled in the organic chemistry art (see Billottet et al., 2006, Oncogene 25:6648-59, and references therein) or obtained from commercial sources, such as Symansis (Auckland, NZ). IC87114 was reported to have an PI3K p110 delta IC₅₀ of 60-500 nM (Chaussade et al., 2007, Biochem. J. 404:449-58; Sadhu et al., 2003, Biochem. Biophys. Res. Comm. 308:764-69; Knight et al., 2006, Cell 125:1-15).

Compound (II) is also known as PIK-39 or 2-([(9H-purin-6-yl)thio]methyl)-5-chloro-3-(2-methoxyphenyl)quinazolin-4(3H)-one. Compound (II) was reported to have an PI3K p110 delta IC₅₀ of 0.18 μM at 10 μM ATP (see Knight et al., 2006, Cell 125:733-47).

Compound (III) is also known as PIK-294 or 2-((4-amino-3-(3-hydroxyphenyl)-1H-pyrazolo[3,4-d]pyrimidin-1-yl)methyl)-5-methyl-3-(o-tolyl)quinazolin-4(3H)-one. Compound (III) was reported to have an PI3K p110 delta IC₅₀ of 0.01 μM at 10 μM ATP (see Knight et al., 2006, Cell 125:733-47).

Compound (IV) is also known as 2-(1-(6-amino-9H-purin-9-yl)ethyl)-3-phenylthieno[3,2-d]pyrimidin-4(3H)-one, Compound (IV) was reported to have an PI3K p110 delta IC₅₀ of 0.04 μM at 10 μM ATP (see International Patent Application Nos. WO 09/064,802 and WO 08/064,018).

Compound (V) is also known as 6-(1-((9H-purin-6-yl)thio)ethyl)-3-methyl-5-phenylisoxazolo[5,4-d]pyrimidin-4(5H)-one. Compound (V) was reported to have an IC₅₀ of 1.2 μM in fMLP-induced elastase exocytosis from neutrophils (see International Patent Application Nos. WO 09/064,802 and WO 08/064,018).

Compound (VI) is also known as 3-isobutyl-6-methyl-4-oxo-N-(pyridin-3-ylmethyl)-3,4-dihydrofuro[2,3-d]pyrimidine-5-carboxamide. Compound (VI) was reported to have an PI3K p110 delta IC₅₀ of 1.2 μM (see International Patent Application No. WO 09/011,617).

Compound (VII) is also known as (S)-5-chloro-N⁴-(1-(8-chloro-2-(pyridin-2-yl)quinolin-3-yl)ethyl)pyrimidine-2,4-diamine. Compound (VII) was reported to have an PI3K p110 delta IC₅₀ of 105 nM (see International Patent Application No. WO 08/118,455).

Compound (VIII) is also known as 4-(2-(1H-indazol-4-yl)-6-((4-(methylsulfonyl)piperazin-1-yl)methyl)thieno[3,2-d]pyrimidin-4-yl)morpholine. Compound (VIII) was reported to have an PI3K p110 delta IC₅₀ of 3 nM at 1 μM ATP (see International Patent Application No. WO 07/129,161; Folkes et al., 2008, J. Med. Chem. 51:5522-32).

Compound (IX) is also known as 4-(6-((4-(azetidin-1-yl)piperidin-1-yl)methyl)-2-(6-fluoro-1H-indol-4-yl)thieno[3,2-d]pyrimidin-4-yl)morpholine.

Compound (X) is also known as 1-((9-ethyl-2-(1H-indol-4-yl)-6-morpholino-9H-purin-8-yl)methyl)-N,N-dimethylpiperidin-4-amine.

Compound (XI) is also known as 4-(2-(1H-indol-4-yl)-6-((4-methylpiperazin-1-yl)methyl)quinazolin-4-yl)morpholine.

Compound (XII) is also known as 4-(2-((hexahydropyrrolo[3,4-c]pyrrol-2(1H)-yl)methyl)-5-(1H-indol-4-yl)thiazolo[4,5-d]pyrimidin-7-yl)morpholine.

Compound (XIII) is also known as N-((4-(6-fluoro-1H-indol-4-yl)-6-morpholinopyrimidin-2-yl)methyl)Nicotinamide.

Compound (XIV) is also known as 6-(6-fluoro-1H-indol-4-yl)-2-morpholino-N-(2-(pyridin-3-yl)ethyl)pyrimidin-4-amine.

Compound (XV) is also known as N-(2-(dimethylamino)ethyl)-4-(6-fluoro-1H-indol-4-yl)-6-morpholinopyrimidine-2-carboxamide.

Compounds (IX)-(XV) were reported as PI3K p110 delta inhibitors (International Patent Application Nos. WO 09/053,715; WO 09/053716; WO 08/125,833; WO 08/125,835; WO 08/125,839; WO 08/152,387; and WO 08/152,390).

Compound (XVI) is also known as 5,5-dimethyl-2-morpholino-5,6-dihydrobenzo[d]thiazol-7(4H)-one. Compound (XVI) was reported to have an PI3K p110 delta IC₅₀ of 0.70 μM (see Alexander et al., 2008, Bioorg. Med. Chem. 18:4316-20).

Compound (XVII) is also shown as (S)-2-(3-((1H-indol-3-yl)methyl)morpholino)-7,7-dimethyl-5,6,7,8-tetrahydro-4H-thiazolo[5,4-c]azepin-4-one.

Compound (XVII) was reported to have an PI3K p110 delta IC₅₀ of 18 nM (see Alexander et al., 2008, Bioorg. Med. Chem. 18:4316-20; International Patent Application No. WO 06/114606).

Compound (XVIII) is also known as 6,6-dimethyl-2-(6-phenyl-2H-benzo[b][1,4]oxazin-4(3H)-yl)-6,7-dihydrothiazolo[5,4-c]pyridin-4(5H)-one. Compound (XVIII) was reported to have an PI3K p110 delta IC₅₀ of 0.05 μM (see Perry et al, 2008, Bioorg. Med. Chem. Lett. 18:4700-04).

Compound (XIX) is also known as 5,5-dimethyl-2-(6-phenyl-2H-benzo[b][1,4]oxazin-4(3H)-yl)-5,6-dihydrobenzo[d]thiazol-7(4H)-one. Compound (XIX) was reported to have an PI3K p110 delta IC₅₀ of 0.08 μM (see Perry et al, 2008, Bioorg. Med. Chem. Lett. 18:4700-04).

Compound (XX) is also known as 6,6-dimethyl-2-(6-(1-methyl-1H-pyrazol-4-yl)-2H-benzo[b][1,4]oxazin-4(3H)-yl)-6,7-dihydrothiazolo[5,4-c]pyridin-4(5H)-one.

Compound (XX) was reported to have an PI3K p110 delta IC₅₀ of 0.03 μM (see Perry et al, 2008, Bioorg. Med. Chem. Lett. 18:4700-04; International Patent Application No. WO 08/001,076).

Compound (XXI) is also known as 2-(6-(1-(2-hydroxy-3-methoxypropyl)-1H-pyrazol-4-yl)-2H-benzo[b][1,4]oxazin-4(3H)-yl)-6,6-dimethyl-6,7-dihydrothiazolo[5,4-c]pyridin-4(5H)-one. Compound (XXI) was reported to have an ED₅₀ of 5 mg/kg in a CD3-induced IL-2 release study in Lewis rats (see Perry et al, 2008, Bioorg. Med. Chem. Lett. 18:5299-302).

Compound (XXII) is also known as 6,6-dimethyl-2-(6-(6-methylpyridazin-3-yl)-2H-benzo[b][1,4]oxazin-4(3H)-yl)-6,7-dihydrothiazolo[5,4-c]pyridin-4(5H)-one.

Compound (XXII) was reported to have an PI3K p110 delta IC₅₀ of 0.05 μM (see Perry et al, 2008, Bioorg, Med. Chem. Lett. 18:4700-04; International Patent Application No. WO 08/001,076).

Compound (XXIII) is also known as ethyl 2-morpholino-7-oxo-4,5,6,7-tetrahydrobenzo[b]thiophene-3-carboxylate.

Compound (XXIV) is also known as (S)-2-(3-((1H-indol-3-yl)methyl)morpholino)-4-methylthiazole-5-carboxamide.

Compound (XXV) is also known as 2-(6-bromo-2H-benzo[b][1,4]oxazin-4(3H)-yl)-5,5-dimethyl-5,6-dihydrobenzo[d]thiazol-7(4H)-one.

Compound (XXVI) is also known as (S)-methyl 3-((4-(6,6-dimethyl-4-oxo-4,5,6,7-tetrahydrothiazolo[5,4-c]pyridin-2-yl)morpholin-3-yl)methyl)-1-methyl-1H-indole-5-carboxylate.

Compounds (XXIII)-(XXVI) were reported as PI3K p110 delta inhibitors (International Patent Application Nos. WO 07/141,504, WO 08/044,022 and WO 08/047,109).

Compound (XXVII) is also known as TG100-115 or 3,3′-(2,4-diaminopteridine-6,7-diyl)diphenol. Compound (XXVII) was reported to have an PI3K p110 delta IC₅₀ of 0.235 μM (see Palanki et al., 2007, J. Med. Chem. 50:4279).

Compound (XXVIII) is also known as TG100-713 or 3-(2,4-diaminopteridin-6-yl)phenol. Compound (XXVIII) was reported to have an PI3K p110 delta IC₅₀ of 24 nM (see Palanki et al., 2007, J. Med. Chem. 50:4279; Doukas et al., 2006, Proc. Nat. Acad. Sci. 103:19866-71).

Compound (XXIX) is also known as TG 100-110 or 6-(1H-indol-4-yl)pteridine-2,4-diamine. Compound (XXIX) was reported to have an PI3K p110 delta IC₅₀ of 0.064 μM (see Palanki et al., 2007, J. Med. Chem. 50:4279; Doukas et al., 2006, Proc. Nat. Acad, Sci. 103:19866-71).

Compound (XXX) or a pharmaceutically acceptable salt thereof may be prepared using methods known to those skilled in the organic chemistry art (see Knight et al., 2010, ACS Med. Chem. Lett. 1:39-43 and references therein) or obtained from commercial sources.

In one embodiment, in Compound (XXX) R¹ is CH and R² is 2,2-(5-thiazolidinyl-2,4-dione)-1-ethylenyl, and the compound useful within the methods of the invention is Compound (XXXI):

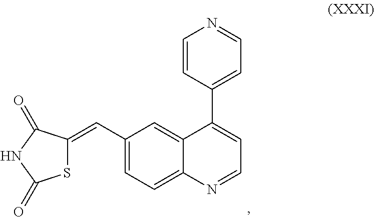 [SMILES: O=C1NC(=O)/C(=C/c2ccc3nccc(-c4ccncc4)c3c2)S1]

also known as (Z)-5-((4-(pyridin-4-yl)quinolin-6-yl)methylene)thiazolidine-2,4-dione, or a pharmaceutically acceptable salt thereof.

In another embodiment, in Compound (XXX) R¹ is N and R² is 2-methoxy-3-(2,4-difluorobenzenosulfonylamino)-pyridin-5-yl, and the compound useful within the methods of the invention is Compound (XXXII):

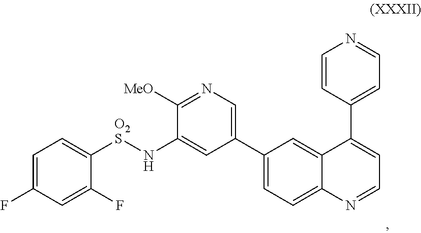 [SMILES: COc1ncc(-c2ccc3nccc(-c4ccncc4)c3c2)cc1NS(=O)(=O)c1ccc(F)cc1F]

also known as 2,4-difluoro-N-(2-methoxy-5-(4-(pyridin-4-yl)quinolin-6-yl)pyridin-3-yl)-benzenesulfonamide, or a pharmaceutically acceptable salt thereof. Compound (XXXII) was reported to have an apparent PI3K p110 delta IC₅₀ of 0.024 nM (Knight et al., 2010, ACS Med. Chem. Lett. 1:39-43)
Salts of the Compounds Useful within the Invention

The compounds useful within the invention may form salts with acids or bases, and such salts are included in the present invention. In one embodiment, the salts are pharmaceutically-acceptable salts. The term “salts” embraces addition salts of free acids or free bases that are compounds useful within the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Examples of pharmaceutically unacceptable acid addition salts include, for example, perchlorates and tetrafluoroborates.

Suitable pharmaceutically acceptable base addition salts of compounds useful within the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared from the corresponding compound by reacting, for example, the appropriate acid or base with the compound.

### Methods of the Invention

In one aspect, the invention includes a method of inhibiting retroviral replication. The method comprises the step of inhibiting phosphoinositide 3 kinase (PI3K) isoform p110 delta in a cell. The step comprises contacting the cell with a pharmaceutically acceptable composition comprising an inhibitor of PI3K p110 delta. In one embodiment, the inhibitor of PI3K p110 delta is a small molecule compound. In another embodiment, the small molecule compound is selected from the group consisting of wortmannin, INK1197, KAR4000 theophylline, CAL-101, CAL-263, Compound (I) (IC87114 or 2-[(6-amino-9H-purin-9-yl)methyl]-5-methyl-3-(o-tolyl)quinazolin-4(3H)-one), Compound (II) (PIK-39 or 2-([(9H-purin-6-yl)thio]methyl)-5-chloro-3-(2-methoxyphenyl)quinazolin-4(3H)-one), Compound (III) (PIK-294 or 2-((4-amino-3-(3-hydroxyphenyl)-1H-pyrazolo[3,4-d]pyrimidin-1-yl)methyl)-5-methyl-3-(o-tolyl)quinazolin-4(3H)-one), Compound (IV) (2-(1-(6-amino-9H-purin-9-yl)ethyl)-3-phenylthieno[3,2-d]pyrimidin-4(3H)-one), Compound (V) (6-(1-((9H-purin-6-yl)thio)ethyl)-3-methyl-5-phenylisoxazolo[5,4-d]pyrimidin-4(5H)-one), Compound (VI) (3-isobutyl-6-methyl-4-oxo-N-(pyridin-3-ylmethyl)-3,4-dihydrofuro[2,3-d]pyrimidine-5-carboxamide), Compound (VII) ((S)-5-chloro-N⁴-(1-(8-chloro-2-(pyridin-2-yl)quinolin-3-yl)ethyl)pyrimidine-2,4-diamine), Compound (VIII) (4-(2-(1H-indazol-4-yl)-6-((4-(methylsulfonyl)piperazin-1-yl)methyl)thieno[3,2-d]pyrimidin-4-yl)morpholine), Compound (IX) (4-(6-((4-(azetidin-1-yl)piperidin-1-yl)methyl)-2-(6-fluoro-1H-indol-4-yl)thieno[3,2-d]pyrimidin-4-yl)morpholine), Compound (X) (1-((9-ethyl-2-(1H-indol-4-yl)-6-morpholino-9H-purin-8-yl)methyl)-N,N-dimethylpiperidin-4-amine), Compound (XI) (4-(2-(1H-indol-4-yl)-6-((4-methylpiperazin-1-yl)methyl)quinazolin-4-yl)morpholine), Compound (XII) (4-(2-((hexahydropyrrolo[3,4-c]pyrrol-2(1H)-yl)methyl)-5-(1H-indol-4-yl)thiazolo[4,5-d]pyrimidin-7-yl)morpholine), Compound (XIII) (N-((4-(6-fluoro-1H-indol-4-yl)-6-morpholinopyrimidin-2-yl)methyl)nicotinamide), Compound (XIV) (6-(6-fluoro-1H-indol-4-yl)-2-morpholino-N-(2-(pyridin-3-yl)ethyl)pyrimidin-4-amine), Compound (XV) (N-(2-(dimethylamino)ethyl)-4-(6-fluoro-1H-indol-4-yl)-6-morpholinopyrimidine-2-carboxamide), Compound (XVI) (5,5-dimethyl-2-morpholino-5,6-dihydrobenzo[d]thiazol-7(4H)-one), Compound (XVII) ((S)-2-(3-((1H-indol-3-yl)methyl)morpholino)-7,7-dimethyl-5,6,7,8-tetrahydro-4H-thiazolo[5,4-c]azepin-4-one), Compound (XVIII) (6,6-dimethyl-2-(6-phenyl-2H-benzo[b][1,4]oxazin-4(3H)-yl)-6,7-dihydrothiazolo[5,4-c]pyridin-4(5H)-one), Compound (XIX) (5,5-dimethyl-2-(6-phenyl-2H-benzo[b][1,4]oxazin-4(3H)-yl)-5,6-dihydrobenzo[d]thiazol-7(4H)-one), Compound (XX) (6,6-dimethyl-2-(6-(1-methyl-1H-pyrazol-4-yl)-2H-benzo[b][1,4]oxazin-4(3H)-yl)-6,7-dihydrothiazolo[5,4-c]pyridin-4(5H)-one), Compound (XXI) (2-(6-(1-(2-hydroxy-3-methoxypropyl)-1H-pyrazol-4-yl)-2H-benzo[b][1,4]oxazin-4(3H)-yl)-6,6-dimethyl-6,7-dihydrothiazolo[5,4-c]pyridin-4(5H)-one), Compound (XXII) (6,6-dimethyl-2-(6-(6-methylpyridazin-3-yl)-2H-benzo[b][1,4]oxazin-4(3H)-yl)-6,7-dihydrothiazolo[5,4-c]pyridin-4(5H)-one), Compound (XXIII) (ethyl 2-morpholino-7-oxo-4,5,6,7-tetrahydrobenzo[b]thiophene-3-carboxylate), Compound (XXIV) ((S)-2-(3-((1H-indol-3-yl)methyl)morpholino)-4-methylthiazole-5-carboxamide), Compound (XXV) (2-(6-bromo-2H-benzo[b][1,4]oxazin-4(3H)-yl)-5,5-dimethyl-5,6-dihydrobenzo[d]thiazol-7(4H)-one), Compound (XXVI) ((S)-methyl 3-((4-(6,6-dimethyl-4-oxo-4,5,6,7-tetrahydrothiazolo[5,4-c]pyridin-2-yl)morpholin-3-yl)methyl)-methyl-1H-indole-5-carboxylate), Compound (XXVII) (TG100-115 or 3,3′-(2,4-diaminopteridine-6,7-diyl)diphenol), Compound (XXVIII) (TG 100-713 or 3-(2,4-diaminopteridin-6-yl)phenol), Compound (XXIX) (TG 100-110 or 6-(1H-indol-4-yl)pteridine-2,4-diamine), Compound (XXX), and a pharmaceutically acceptable salt thereof,

wherein in Compound (XXX) R¹ is N or CH, and R² is a substituent selected from the group consisting of 2,2-(5-thiazolidinyl-2,4-dione)-1-ethylenyl; phenyl; pyridin-2-yl; 1H-indaz-5-yl; 1H-pyrazolo[3,4-b]pyridin-5-yl; 2-amino-3-sulfonamido-pyridin-5-yl; 2-amino-3-[(N-2,4-difluorophenyl)sulfonamido]-pyridin-5-yl; 3-[(N-2,4-difluorophenyl)sulfonamide]-pyridin-5-yl; 3-(2,4-difluorobenzenosulfonylamino)-pyridin-5-yl; and 2-methoxy-3-(2,4-difluorobenzenosulfonylamino)-pyridin-5-yl.

In another aspect, the invention includes a method of inhibiting retroviral pathogenesis. The method comprises the step of inhibiting phosphoinositide 3 kinase (PI3K) isoform p110 delta in a cell. The step comprises contacting the cell with a pharmaceutically acceptable composition comprising an inhibitor of PI3K p110 delta. In one embodiment, the inhibitor of PI3K p110 delta is a small molecule compound. In another embodiment, the small molecule compound is selected from the group consisting of wortmannin, INK197, KAR4000 theophylline, CAL-101, CAL-263, Compound (I) (IC87114 or 2-[(6-amino-9H-purin-9-yl)methyl]-5-methyl-3-(o-tolyl)quinazolin-4(3H)-one), Compound (II) (PIK-39 or 2-([(9H-purin-6-yl)thio]methyl)-5-chloro-3-(2-methoxyphenyl)quinazolin-4(3H)-one), Compound (III) (PIK-294 or 2-((4-amino-3-(3-hydroxyphenyl)-1H-pyrazolo[3,4-d]pyrimidin-1-yl)methyl)-5-methyl-3-(o-tolyl)quinazolin-4(3H)-one), Compound (IV) (2-(1-(6-amino-9H-purin-9-yl)ethyl)-3-phenylthieno[3,2-d]pyrimidin-4(3H)-one), Compound (V) (6-(1-((9H-purin-6-yl)thio)ethyl)-3-methyl-5-phenylisoxazolo[5,4-d]pyrimidin-4(5H)-one), Compound (VI) (3-isobutyl-6-methyl-4-oxo-N-(pyridin-3-ylmethyl)-3,4-dihydrofuro[2,3-d]pyrimidine-5-carboxamide), Compound (VII) ((S)-5-chloro-N⁴-(1-(8-chloro-2-(pyridin-2-yl)quinolin-3-yl)ethyl)pyrimidine-2,4-diamine), Compound (VIII) (4-(2-(1H-indazol-4-yl)-6-((4-(methylsulfonyl)piperazin-1-yl)methyl)thieno[3,2-d]pyrimidin-4-yl)morpholine), Compound (IX) (4-(6-((4-(azetidin-1-yl)piperidin-1-yl)methyl)-2-(6-fluoro-1H-indol-4-yl)thieno[3,2-d]pyrimidin-4-yl)morpholine), Compound (X) (1-((9-ethyl-2-(1H-indol-4-yl)-6-morpholino-9H-purin-8-yl)methyl)-N,N-dimethylpiperidin-4-amine), Compound (XI) (4-(2-(1H-indol-4-yl)-6-((4-methylpiperazin-1-yl)methyl)quinazolin-4-yl)morpholine), Compound (XII) (4-(2-((hexahydropyrrolo[3,4-c]pyrrol-2(1H)-yl)methyl)-5-(1H-indol-4-yl)thiazolo[4,5-d]pyrimidin-7-yl)morpholine), Compound (XIII) (N-((4-(6-fluoro-1H-indol-4-yl)-6-morpholinopyrimidin-2-yl)methyl)nicotinamide), Compound (XIV) (6-(6-fluoro-1H-indol-4-yl)-2-morpholino-N-(2-(pyridin-3-yl)ethyl)pyrimidin-4-amine), Compound (XV) (N-(2-(dimethylamino)ethyl)-4-(6-fluoro-1H-indol-4-yl)-6-morpholinopyrimidine-2-carboxamide), Compound (XVI) (5,5-dimethyl-2-morpholino-5,6-dihydrobenzo[d]thiazol-7(4H)-one), Compound (XVII) ((S)-2-(3-((1H-indol-3-yl)methyl)morpholino)-7,7-dimethyl-5,6,7,8-tetrahydro-4H-thiazolo[5,4-c]azepin-4-one), Compound (XVIII) (6,6-dimethyl-2-(6-phenyl-2H-benzo[b][1,4]oxazin-4(3H)-yl)-6,7-dihydrothiazolo[5,4-c]pyridin-4(5H)-one), Compound (XIX) (5,5-dimethyl-2-(6-phenyl-2H-benzo[b][1,4]oxazin-4(3H)-yl)-5,6-dihydrobenzo[d]thiazol-7(4H)-one), Compound (XX) (6,6-dimethyl-2-(6-(1-methyl-1H-pyrazol-4-yl)-2H-benzo[b][1,4]oxazin-4(3H)-yl)-6,7-dihydrothiazolo[5,4-c]pyridin-4(5H)-one), Compound (XXI) (2-(6-(1-(2-hydroxy-3-methoxypropyl)-1H-pyrazol-4-yl)-2H-benzo[b][1,4]oxazin-4(3H)-yl)-6,6-dimethyl-6,7-dihydrothiazolo[5,4-c]pyridin-4(5H)-one), Compound (XXII) (6,6-dimethyl-2-(6-(6-methylpyridazin-3-yl)-2H-benzo[b][1,4]oxazin-4(3H)-yl)-6,7-dihydrothiazolo[5,4-c]pyridin-4(5H)-one), Compound (XXIII) (ethyl 2-morpholino-7-oxo-4,5,6,7-tetrahydrobenzo[b]thiophene-3-carboxylate), Compound (XXIV) ((S)-2-(3-((1H-indol-3-yl)methyl)morpholino)-4-methylthiazole-5-carboxamide), Compound (XXV) (2-(6-bromo-2H-benzo[b][1,4]oxazin-4(3H)-yl)-5,5-dimethyl-5,6-dihydrobenzo[d]thiazol-7(4H)-one), Compound (XXVI) ((S)-methyl 3-((4-(6,6-dimethyl-4-oxo-4,5,6,7-tetrahydrothiazolo[5,4-c]pyridin-2-yl)morpholin-3-yl)methyl)-1-methyl-1H-indole-5-carboxylate), Compound (XXVII) (TG100-115 or 3,3′-(2,4-diaminopteridine-6,7-diyl)diphenol), Compound (XXVIII) (TG100-713 or 3-(2,4-diaminopteridin-6-yl)phenol), Compound (XXIX) (TG 100-110 or 6-(1H-indol-4-yl)pteridine-2,4-diamine), Compound (XXX), and a pharmaceutically acceptable salt thereof,

wherein in Compound (XXX) R¹ is N or CH, and R² is a substituent selected from the group consisting of 2,2-(5-thiazolidinyl-2,4-dione)-1-ethylenyl; phenyl; pyridin-2-yl; 1H-indaz-5-yl; 1H-pyrazolo[3,4-b]pyridin-5-yl; 2-amino-3-sulfonamido-pyridin-5-yl; 2-amino-3-[(N-2,4-difluorophenyl)sulfonamido]-pyridin-5-yl; 3-[(N-2,4-difluorophenyl)sulfonamide]-pyridin-5-yl; 3-(2,4-difluorobenzenosulfonylamino)-pyridin-5-yl; and 2-methoxy-3-(2,4-difluorobenzenosulfonylamino)-pyridin-5-yl.

In yet another aspect, the invention includes a method of treating or preventing retroviral infection in a mammal. The method comprises the step of administering an effective amount of a composition comprising an inhibitor of phosphoinositide 3 kinase (PI3K) isoform p110 delta to the mammal in need thereof, wherein the inhibitor interferes with PI3K p110 delta activation and retroviral replication. In one embodiment, the inhibitor of PI3K p110 delta is a small molecule compound. In another embodiment, the small molecule compound is selected from the group consisting of wortmannin, INK1197, KAR4000 theophylline, CAL-101, CAL-263, Compound (I) (IC87114 or 2-[(6-amino-9H-purin-9-yl)methyl]-5-methyl-3-(o-tolyl)quinazolin-4(3H)-one), Compound (II) (PIK-39 or 2-([(9H-purin-6-yl)thio]methyl)-5-chloro-3-(2-methoxyphenyl)quinazolin-4(3H)-one), Compound (III) (PIK-294 or 2-((4-amino-3-(3-hydroxyphenyl)-1H-pyrazolo[3,4-d]pyrimidin-1-yl)methyl)-5-methyl-3-(o-tolyl)quinazolin-4(3H)-one), Compound (IV) (2-(1-(6-amino-9H-purin-9-yl)ethyl)-3-phenylthieno[3,2-d]pyrimidin-4(3H)-one), Compound (V) (6-(1-((9H-purin-6-yl)thio)ethyl)-3-methyl-5-phenylisoxazolo[5,4-d]pyrimidin-4(5H)-one), Compound (VI) (3-isobutyl-6-methyl-4-oxo-N-(pyridin-3-ylmethyl)-3,4-dihydrofuro[2,3-d]pyrimidine-5-carboxamide), Compound (VII) ((S)-5-chloro-N⁴-(1-(8-chloro-2-(pyridin-2-yl)quinolin-3-yl)ethyl)pyrimidine-2,4-diamine), Compound (VIII) (4-(2-(1H-indazol-4-yl)-6-((4-(methylsulfonyl)piperazin-1-yl)methyl)thieno[3,2-d]pyrimidin-4-yl)morpholine), Compound (IX) (4-(6-((4-(azetidin-1-yl)piperidin-1-yl)methyl)-2-(6-fluoro-1H-indol-4-yl)thieno[3,2-d]pyrimidin-4-yl)morpholine), Compound (X) (1-((9-ethyl-2-(1H-indol-4-yl)-6-morpholino-9H-purin-8-yl)methyl)-N,N-dimethylpiperidin-4-amine), Compound (XI) (4-(2-(1H-indol-4-yl)-6-((4-methylpiperazin-1-yl)methyl)quinazolin-4-yl)morpholine), Compound (XII) (4-(2-((hexahydropyrrolo[3,4-c]pyrrol-2(1H)-yl)methyl)-5-(1H-indol-4-yl)thiazolo[4,5-d]pyrimidin-7-yl)morpholine), Compound (XIII) (N-((4-(6-fluoro-1H-indol-4-yl)-6-morpholinopyrimidin-2-yl)methyl)nicotinamide), Compound (XIV) (6-(6-fluoro-1H-indol-4-yl)-2-morpholino-N-(2-(pyridin-3-yl)ethyl)pyrimidin-4-amine), Compound (XV) (N-(2-(dimethylamino)ethyl)-4-(6-fluoro-1H-indol-4-yl)-6-morpholinopyrimidine-2-carboxamide), Compound (XVI) (5,5-dimethyl-2-morpholino-5,6-dihydrobenzo[d]thiazol-7(4H)-one), Compound (XVII) ((S)-2-(3-((1H-indol-3-yl)methyl)morpholino)-7,7-dimethyl-5,6,7,8-tetrahydro-4H-thiazolo[5,4-c]azepin-4-one), Compound (XVIII) (6,6-dimethyl-2-(6-phenyl-2H-benzo[b][1,4]oxazin-4(3H)-yl)-6,7-dihydrothiazolo[5,4-c]pyridin-4(5H)-one), Compound (XIX) (5,5-dimethyl-2-(6-phenyl-2H-benzo[b][1,4]oxazin-4(3H)-yl)-5,6-dihydrobenzo[d]thiazol-7(4H)-one), Compound (XX) (6,6-dimethyl-2-(6-(1-methyl-1H-pyrazol-4-yl)-2H-benzo[b][1,4]oxazin-4(3H)-yl)-6,7-dihydrothiazolo[5,4-c]pyridin-4(5H)-one), Compound (XXI) (2-(6-(1-(2-hydroxy-3-methoxypropyl)-1H-pyrazol-4-yl)-2H-benzo[b][1,4]oxazin-4(3H)-yl)-6,6-dimethyl-6,7-dihydrothiazolo[5,4-c]pyridin-4(5H)-one), Compound (XXII) (6,6-dimethyl-2-(6-(6-methylpyridazin-3-yl)-2H-benzo[b][1,4]oxazin-4(3H)-yl)-6,7-dihydrothiazolo[5,4-c]pyridin-4(5H)-one), Compound (XXIII) (ethyl 2-morpholino-7-oxo-4,5,6,7-tetrahydrobenzo[b]thiophene-3-carboxylate), Compound (XXIV) ((S)-2-(3-((1H-indol-3-yl)methyl)morpholino)-4-methylthiazole-5-carboxamide), Compound (XXV) (2-(6-bromo-2H-benzo[b][1,4]oxazin-4(3H)-yl)-5,5-dimethyl-5,6-dihydrobenzo[d]thiazol-7(4H)-one), Compound (XXVI) ((S)-methyl 3-((4-(6,6-dimethyl-4-oxo-4,5,6,7-tetrahydrothiazolo[5,4-c]pyridin-2-yl)morpholin-3-yl)methyl)-1-methyl-1H-indole-5-carboxylate), Compound (XXVII) (TG 100-115 or 3,3′-(2,4-diaminopteridine-6,7-diyl)diphenol), Compound (XXVIII) (TG 100-713 or 3-(2,4-diaminopteridin-6-yl)phenol), Compound (XXIX) (TG 100-110 or 6-(1H-indol-4-yl)pteridine-2,4-diamine), Compound (XXX), and a pharmaceutically acceptable salt thereof,

wherein in Compound (XXX) R¹ is N or CH, and R² is a substituent selected from the group consisting of 2,2-(5-thiazolidinyl-2,4-dione)-1-ethylenyl; phenyl; pyridin-2-yl; 1H-indaz-5-yl; 1H-pyrazolo[3,4-b]pyridin-5-yl; 2-amino-3-sulfonamido-pyridin-5-yl; 2-amino-3-[(N-2,4-difluorophenyl)sulfonamido]-pyridin-5-yl; 3-[(N-2,4-difluorophenyl)sulfonamide]-pyridin-5-yl; 3-(2,4-difluorobenzenosulfonylamino)-pyridin-5-yl; and 2-methoxy-3-(2,4-difluorobenzenosulfonylamino)-pyridin-5-yl.

In one embodiment, the composition further comprises at least one anti-HIV drug. In another embodiment, the at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, and protease inhibitors.

In one embodiment, the composition further comprises at least one immunomodulator. In another embodiment, the at least one immunomodulator is an anti-inflammatory agent. In yet another embodiment, the anti-inflammatory agent is non-steroidal. In yet another embodiment, the anti-inflammatory agent is a non-steroidal anti-inflammatory (NSAID) agent.

In one embodiment, the retrovirus is HIV. In another embodiment, the subject is a mammal. In yet another embodiment, the subject is human.

### Composition

As described elsewhere herein, the invention is based on the discovery that inhibition of PI3K p110 delta may provide a therapeutic benefit by inhibiting retroviral infection. The invention comprises compositions and methods for modulating PI3K p110 delta in a cell thereby inhibiting the PI3K p110 delta response in the cell.

Based on the disclosure herein, the present invention includes a generic concept for inhibiting PI3K pt 10 delta or PI3K p110 delta signaling pathway in a cell of a mammal suffering from, or at risk of, retroviral infection.

In one embodiment, the invention comprises a composition for inhibiting PI3K p110 delta. The composition comprises an inhibitor of one or more of the following: PI3K p110 delta or PI3K p110 delta down stream signaling pathway in a cell. Thus, as referred to herein, inhibiting PI3K p110 delta may also encompass inhibiting any component of the PI3K p110 delta signaling pathway.

The composition comprising the inhibitor of a component of the PI3K p110 delta signaling pathway may be any type of inhibitor. For example and without limitation, the inhibitor may be selected from the group consisting of a small interfering RNA (siRNA), a microRNA, an antisense nucleic acid, a ribozyme, an expression vector encoding a transdominant negative mutant, an intracellular antibody, a peptide and a small molecule compound.

As disclosed herein, the inhibition of a component of the PI3K p110 delta signaling pathway in a cell inhibits retroviral infection in the cell. These effects are mediated through inhibition of PI3K p110 delta signaling pathway. One skilled in the art will appreciate, based on the disclosure provided herein, that one way to decrease the mRNA and/or protein levels of a component of the PI3K p110 delta signaling pathway in a cell is by reducing or inhibiting expression of the nucleic acid encoding a desired component of the PI3K p110 delta signaling pathway. Thus, the protein level of the component of the PI3K p110 delta signaling pathway in a cell may also be decreased using a molecule or compound that inhibits or reduces gene expression such as, for example, an antisense molecule or a ribozyme.

By way of a non-limited example, inhibition of a component of PI3K p110 delta signaling pathway is described below in the context of decreasing the mRNA and/or protein levels of a component of the PI3K p110 delta signaling pathway in a cell by reducing or inhibiting expression of the nucleic acid encoding a desired component of the PI3K p110 delta signaling pathway.

In a preferred embodiment, the modulating sequence is an antisense nucleic acid sequence that is expressed by a plasmid vector. The antisense expressing vector is used to transfect a mammalian cell or the mammal itself, thereby causing reduced endogenous expression of a desired component of the PI3K p110 delta signaling pathway in the cell. However, the invention should not be construed to be limited to inhibiting expression of a component of the PI3K p110 delta signaling pathway by transfection of cells with antisense molecules. Rather, the invention encompasses other methods known in the art for inhibiting expression or activity of a protein in the cell including, but not limited to, the use of a ribozyme, the expression of a non-functional component of the PI3K p110 delta signaling pathway (i.e. transdominant negative mutant) and use of an intracellular antibody.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific Amerimay 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (Anal. Biochem. 1988, 172:289). Such antisense molecules may be provided to the cell via genetic expression using DNA encoding the antisense molecule as taught by U.S. Pat. No. 5,190,931 by Inoue.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

The ability to specifically inhibit gene function in a variety of organisms utilizing antisense RNA or dsRNA-mediated interference (RNAi or dsRNA) is well known in the fields of molecular biology. dsRNA (RNAi) typically comprises a polynucleotide sequence identical or homologous to a target gene (or fragment thereof) linked directly, or indirectly, to a polynucleotide sequence complementary to the sequence of the target gene (or fragment thereof). The dsRNA may comprise a polynucleotide linker sequence of sufficient length to allow for the two polynucleotide sequences to fold over and hybridize to each other; however, a linker sequence is not necessary. The linker sequence is designed to separate the antisense and sense strands of RNAi significantly enough to limit the effects of steric hindrances and allow for the formation of dsRNA molecules and should not hybridize with sequences within the hybridizing portions of the dsRNA molecule. The specificity of this gene silencing mechanism appears to be extremely high, blocking expression only of targeted genes, while leaving other genes unaffected. Accordingly, one method for treating retroviral infection according to the invention comprises the use of materials and methods utilizing double-stranded interfering RNA (dsRNAi), or RNA-mediated interference (RNAi) comprising polynucleotide sequences identical or homologous to a desired component of TGF-β signaling pathway. The terms “dsRNAi”, “RNAi”, “iRNA”, and “siRNA” are used interchangeably herein unless otherwise noted.

RNA containing a nucleotide sequence identical to a fragment of the target gene is preferred for inhibition; however, RNA sequences with insertions, deletions, and point mutations relative to the target sequence may also be used for inhibition. Sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Alternatively, the duplex region of the RNA may be defined functionally as a nucleotide sequence that is capable of hybridizing with a fragment of the target gene transcript.

RNA may be synthesized either in vivo or in vitro. Endogenous RNA polymerase of the cell may mediate transcription in vivo, or cloned RNA polymerase may be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands); the promoters may be known inducible promoters such as baculovirus. Inhibition may be targeted by specific transcription in an organ, tissue, or cell type. The RNA strands may or may not be polyadenylated; the RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (see, for example, International Application No. WO 97/32016; U.S. Pat. Nos. 5,593,874; 5,698,425; 5,712,135; 5,789,214; and 5,804,693; and the references cited therein). If synthesized chemically or by in vitro enzymatic synthesis, the RNA may be purified prior to introduction into the cell. For example, RNA may be purified from a mixture by extraction with a solvent or resin, precipitation, electrophoresis, chromatography, or a combination thereof. Alternatively, the RNA may be used with no, or a minimum of, purification to avoid losses due to sample processing. The RNA may be dried for storage or dissolved in an aqueous solution. The solution may contain buffers or salts to promote annealing, and/or stabilization of the duplex strands.

Fragments of genes may also be utilized for targeted suppression of gene expression. These fragments are typically in the approximate size range of about 20 consecutive nucleotides of a target sequence. Thus, targeted fragments are preferably at least about 15 consecutive nucleotides. In certain embodiments, the gene fragment targeted by the RNAi molecule is about 20-25 consecutive nucleotides in length. In a more preferred embodiment, the gene fragments are at least about 25 consecutive nucleotides in length. In an even more preferred embodiment, the gene fragments are at least 50 consecutive nucleotides in length. Various embodiments also allow for the joining of one or more gene fragments of at least about 15 nucleotides via linkers. Thus, RNAi molecules useful in the practice of the instant invention may contain any number of gene fragments joined by linker sequences.

In yet other embodiments, the invention includes full length or fragments of p110 delta. The gene fragments may range from one nucleotide less than the full-length gene. Nucleotide sequences for PI3K p110 delta and components of PI3K p110 delta signaling pathway are known in the art and may be obtained from patent publications, public databases containing nucleic acid sequences, or commercial vendors. A skilled artisan would understand that the disclosure presented herein provides sufficient written support for any fragment length ranging from about 15 consecutive polynucleotides to one nucleotide less than the full length polynucleotide sequence of PI3K p110 delta and components of PI3K p110 delta signaling pathway may have a whole number value ranging from about 15 consecutive nucleotides to one nucleotide less than the full length polynucleotide.

Accordingly, methods utilizing RNAi molecules in the practice of the subject invention are not limited to those that are targeted to the full-length polynucleotide or gene. Gene product may be inhibited with an RNAi molecule that is targeted to a portion or fragment of the exemplified polynucleotides; high homology (90-95%) or greater identity is also preferred, but not essential, for such applications.

In another aspect of the invention, the dsRNA molecules of the invention may be introduced into cells with single stranded (ss) RNA molecules that are sense or anti-sense RNA derived from the nucleotide sequences disclosed herein. Methods of introducing ssRNA and dsRNA molecules into cells are well-known to the skilled artisan and includes transcription of plasmids, vectors, or genetic constructs encoding the ssRNA or dsRNA molecules according to this aspect of the invention; electroporation, biolistics, or other well-known methods of introducing nucleic acids into cells may also be used to introduce the ssRNA and dsRNA molecules of this invention into cells.

Other types of gene inhibition technology may be used to inhibit PI3K p110 delta and/or components of PI3K p110 delta signaling pathway in a cell. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules may be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences that are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of a component of PI3K p110 delta signaling pathway may be designed by incorporating target sequences into the basic ribozyme structure that are complementary to the mRNA sequence of the desired component of PI3K p110 delta signaling pathway of the present invention. Ribozymes targeting the desired component of PI3K p110 delta signaling pathway may be synthesized using commercially available reagents (Applied Biosystems, Inc., Foster City, Calif.) or they may be genetically expressed from DNA encoding them.

In another aspect of the invention, the component of the PI3K p110 delta signaling pathway may be inhibited by way of inactivating and/or sequestering the desired component of the PI3K p110 delta signaling pathway. As such, inhibiting the effects of a component of the PI3K p110 delta signaling pathway may be accomplished by using a transdominant negative mutant. Alternatively an intracellular antibody specific for the desired component of the PI3K p110 delta signaling pathway, otherwise known as an antagonist to the component of the PI3K p110 delta signaling pathway may be used. In one embodiment, the antagonist is a protein and/or compound having the desirable property of interacting with a binding partner of the component of the PI3K p110 delta signaling pathway and thereby competing with the corresponding wild-type component of the PI3K p110 delta signaling pathway. In another embodiment, the antagonist is a protein and/or compound having the desirable property of interacting with the component of the PI3K p110 delta signaling pathway and thereby sequestering the component of the PI3K p110 delta signaling pathway.

By way of a non-limited example, an antibody is described below as an example of inactivating and/or sequestering the desired component of the PI3K p110 delta signaling pathway.

### Antibodies

As will be understood by one skilled in the art, any antibody that may recognize and specifically bind to PI3K p110 delta or a component involved in PI3K p110 delta signaling pathway is useful in the present invention. The invention should not be construed to be limited to any one type of antibody, either known or heretofore unknown, provided that the antibody may specifically bind to a component involved in PI3K p110 delta signaling pathway. Methods of making and using such antibodies are well known in the art. For example, the generation of polyclonal antibodies may be accomplished by inoculating the desired animal with the antigen and isolating antibodies which specifically bind the antigen therefrom. Monoclonal antibodies directed against full length or peptide fragments of a protein or peptide may be prepared using any well known monoclonal antibody preparation procedures, such as those described, for example, in Harlow et al. (1989, Antibodies, A Laboratory Manual, Cold Spring Harbor, N.Y.) and in Tuszynski et al. (1988, Blood 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein. However, the invention should not be construed as being limited solely to methods and compositions including these antibodies, but should be construed to include other antibodies, as that term is defined elsewhere herein.

In some instances, it is desirable to prepare monoclonal antibodies from various mammalian hosts, such as rodents (e.g., mice), primates (e.g., humans), etc. Descriptions of techniques for preparing such monoclonal antibodies are well known and are described, for example, in Harlow et al., ANTIBODIES: A LABORATORY MANUAL, COLD SPRING HARBOR LABORATORY, Cold Spring Harbor, N.Y. (1988); Harlow et al., USING ANTIBODIES: A LABORATORY MANUAL, (Cold Spring Harbor Press, New York, 1998); Breitling et al., RECOMBINANT ANTIBODIES (Wiley-Spektrum, 1999); and Kohler et al., 1997 Nature 256: 495-497; U.S. Pat. Nos. 5,693,762; 5,693,761; 5,585,089; and 6,180,370.

Nucleic acid encoding an antibody obtained using the procedures described herein may be cloned and sequenced using technology that is available in the art, and is described, for example, in Wright et al. (Critical Rev. in Immunol. 1992, 12:125-168) and the references cited therein. Further, the antibody of the invention may be “humanized” using the technology described in Wright et al. (supra) and in the references cited therein, and in Gu et al. (Thrombosis and Hematocyst 1997, 77:755-759).

Alternatively, antibodies may be generated using phage display technology. To generate a phage antibody library, a cDNA library is first obtained from mRNA that is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage that encode the desired antibody may be engineered such that the protein is displayed on the surface thereof in such a manner that it is available for binding to its corresponding binding protein, e.g., the antigen against which the antibody is directed. Thus, when bacteriophage that express a specific antibody are incubated in the presence of a cell that expresses the corresponding antigen, the bacteriophage will bind to the cell. Bacteriophage that do not express the antibody will not bind to the cell. Such panning techniques are well known in the art and are described for example, in Wright et al. (Critical Rev. in Immunol. 1992, 12:125-168).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage that encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks er al. (1991, J Mol Biol 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J Mol Biol 248:97-105).

The invention encompasses polyclonal, monoclonal, synthetic antibodies, and the like. One skilled in the art would understand, based upon the disclosure provided herein, that the crucial feature of the antibody of the invention is that the antibody specifically bind with a component involved in PI3K p110 delta signaling pathway.

### Vectors

In other related aspects, the invention includes an isolated nucleic acid encoding an inhibitor of the invention, operably linked to a nucleic acid comprising a promoter/regulatory sequence such that the nucleic acid is preferably capable of directing expression of the inhibitor encoded by the nucleic acid. Thus, the invention encompasses expression vectors and methods for the introduction of exogenous DNA into cells with concomitant expression of the exogenous DNA in the cells such as those described, for example, in Sambrook et al. (200, Molecular Cloning: A Laboratory Manual, Cold Spring Harbor Laboratory, New York), and in Ausubel et al. (1997, Current Protocols in Molecular Biology, John Wiley & Sons, New York).

In another aspect, the invention includes a vector comprising an siRNA polynucleotide. Preferably, the siRNA polynucleotide is capable of inhibiting the expression of a target polypeptide, wherein the target polypeptide is p110 delta. The incorporation of a desired polynucleotide into a vector and the choice of vectors is well-known in the art as described in, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems may be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., International Applications Nos. WO 01/96584 and WO 01/29058; and U.S. Pat. No. 6,326,193.

For expression of the desired inhibitor of p110 delta, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements may be increased to 50 bp apart before activity begins to decline. Depending on the promoter, it appears that individual elements may function either co-operatively or independently to activate transcription.

A promoter may be one naturally associated with a gene or polynucleotide sequence, as may be obtained by isolating the 5′ non-coding sequences located upstream of the coding segment and/or exon. Such a promoter may be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, may be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et at. (2001, Molecular Cloning: A Laboratory Manual, Cold Spring Harbor Laboratory, New York). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the desired inhibitor of p110 delta, the expression vector to be introduced into a cell may also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector may be readily introduced into a host cell, e.g., mammalian, bacterial, yeast or insect cell by any method in the art. For example, the expression vector may be transferred into a host cell by physical, chemical or biological means.

Physical methods for introducing a polynucleotide into a host cell include calcium phosphate precipitation, lipofection, particle bombardment, microinjection, eleectroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors may be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

To generate a genetically modified cell, any DNA vector or delivery vehicle may be utilized to transfer the desired PI3K p110 delta inhibitor polynucleotide to a cell in vitro or in vivo. In the case where a non-viral delivery system is utilized, a preferred delivery vehicle is a liposome. The above-mentioned delivery systems and protocols therefore may be found in Gene Targeting Protocols, 2ed., pp 1-35 (2002) and Gene Transfer and Expression Protocols, Vol. 7, Murray ed., pp 81-89 (1991).

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

PI3K p110 Delta Inhibitor

In addition to a genetic approach, the invention includes the use of small molecule compounds to inhibit PI3K p110 delta, a component of the PI3K p110 delta signaling pathway, or any combination thereof. By way of a non-limiting example, IC87114, a selective inhibitor of PI3K p110 delta is useful in inhibiting PI3K p110 delta signaling pathway in a cell. The disclosure presented herein demonstrates that PI3K p110 delta inhibitors are able to inhibit PI3K p110 delta, a component of the PI3K p110 delta signaling pathway, or a combination thereof, to provide a therapeutic benefit in infected mammals. For example, the PI3K p110 delta inhibitor in the form of a small molecule compound may significantly reduced viral loads of infected mammals. In addition, the PI3K p110 delta inhibitor is able to reduce the number of cellular infiltration compared to a mammal not treated with the inhibitor. Also, the treatment with the inhibitor reduces the number of inflammatory cells infiltrating the cells of infected mammals. Thus, the inhibitor of the invention provides a means to regulate retroviral replication and pathogenesis. That is, any inhibitor of the invention that may therapeutically target PI3K p110 delta provides a therapy against retroviral infection. Thus, both genetic and pharmacologic means of PI3K p110 delta signaling inhibition is included in the invention as a useful strategy against retroviral infection.

### Combinational Therapy

In one aspect, the compositions of the invention relating to inhibiting p110 delta, a component of PI3K p110 delta signaling pathway, or any combinations thereof, may be combined with one or more immunomodulators. A preferred composition has an effective amount of a PI3K p110 delta inhibitor to inhibit or reduce retroviral infection in combination with an effective amount of one or more, anti-inflammatory agents, preferably non-steroidal anti-inflammatory agents to reduce inflammatory responses in the subject.

Immunomodulators include immune suppressors or enhancers and anti-inflammatory agents. Preferred immunomodulators are anti-inflammatory agents. The anti-inflammatory agent may be non-steroidal, steroidal, or a combination thereof.

Preferred anti-inflammatory agents are non-steroidal anti-inflammatory (NSAID) agents. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

In one embodiment, immunomodulators are COX-2 inhibitors such as celecoxib and aminosalicylate drugs such as mesalazine and sulfasalazine. In a preferred embodiment, the disclosed composition contains an effective amount of an inhibitor of PI3K p110 delta to inhibit or reduce retroviral infection in a subject in combination with an effective amount of celecoxib and mesalazine to reduce inflammatory responses in the subject.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, predisone, beclomethasone dipropionate, triamcinolone, and mixtures thereof.

In another aspect, the compounds useful within the methods of the invention may be used in combination with one or more additional compounds useful for treating HIV infections. These additional compounds may comprise compounds that are commercially available or synthetically accessible to those skilled in the art. These additional compounds are known to treat, prevent, or reduce the symptoms of HIV infections.

In non-limiting examples, the compounds useful within the invention may be used in combination with one or more of the following anti-HIV drugs:

HIV Combination Drugs: efavirenz, emtricitabine or tenofovir disoproxil fumarate (Atripla®/BMS, Gilead); lamivudine or zidovudine (Combivir®/GSK); abacavir or lamivudine (Epzicom®/GSK); abacavir, lamivudine or zidovudine (Trizivir®/GSK); emtricitabine, tenofovir disoproxil fiumarate (Truvada®/Gilead).

Entry and Fusion Inhibitors: maraviroc (Celsentri®, Selzenty®/Pfizer); pentafuside or enfuvirtide (Fuzeon®/Roche, Trimeris).

Integrase Inhibitors: raltegravir or MK-0518 (Isentress®/Merck).

Non-Nucleoside Reverse Transcriptase Inhibitors: delavirdine mesylate or delavirdine (Rescriptor®/Pfizer); nevirapine (Viramune®/Boehringer Ingelheim); stocrin or efavirenz (Sustiva®/BMS); etravirine (Intelence®/Tibotec).

Nucleoside Reverse Transcriptase Inhibitors: lamivudine or 3TC (Epivir®/GSK); FTC, emtricitabina or coviracil (Emtriva®/Gilead); abacavir (Ziagen®/GSK); zidovudina, ZDV, azidothymidine or AZT (Retrovir®/GSK); ddI, dideoxyinosine or didanosine (Videx®/BMS); abacavir sulfate plus lamivudine (Epzicom®/GSK); stavudine, d4T, or estavudina (Zerit®/BMS); tenofovir, PMPA prodrug, or tenofovir disoproxil filmarate (Viread®/Gilead).

Protease Inhibitors: amprenavir (Agenerase®/GSK, Vertex); atazanavir (Reyataz®/BMS); tipranavir (Aptivus®/Boehringer Ingelheim); darunavir (Prezist®/Tibotec); fosamprenavir (Telzir®, Lexiva®/GSK, Vertex); indinavir sulfate (Crixivan®/Merck); saquinavir mesylate (Invirase®/Roche); lopinavir or ritonavir (Kaletra®/Abbott); nelfinavir mesylate (Viracept®/Pfizer); ritonavir (Norvir®/Abbott).

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Therapeutic Application

The present invention includes an inhibitor of p110 delta, a component of PI3K p110 delta signaling pathway, or any combinations thereof. The invention also includes a cell having heighted anti-retroviral activity compared to an otherwise identical cell not treated according to the present invention.

The present invention envisions treating a disorder or symptoms associated with retroviral infection in a mammal by the administration to the mammal in need thereof a composition of the invention, e.g. an inhibitor of p110 delta, a component of PI3K p110 delta signaling pathway, or any combinations thereof. The mammal is preferably a human.

In one embodiment, the present invention provides a method of treating a disease, disorder, or condition associated with a retroviral infection. Preferably, the retroviral infection is HIV. The method comprises administering a mammal in need thereof a therapeutically effective amount of a pharmaceutical composition comprising an inhibitor of p110 delta, an inhibitor of a component of p110 delta, or any combination thereof.

### Administration/Dosage/Formulations

When the compositions of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” carrier, diluent, excipient, and/or salt is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention may be prepared by procedures known in the art using well known and readily available ingredients. The therapeutic agents of the invention may also be formulated as solutions appropriate for parenteral administration, for instance by intramuscular, subcutaneous or intravenous routes.

The expression vectors, transduced cells, polynucleotides and polypeptides (active ingredients) of this invention may be formulated and administered to treat a variety of disease states by any means that produces contact of the active ingredient with the agent's site of action in the body of the organism. They may be administered by any conventional means available for use in conjunction with pharmaceuticals, either as individual therapeutic active ingredients or in a combination of therapeutic active ingredients. They may be administered alone, but are generally administered with a pharmaceutical carrier selected on the basis of the chosen route of administration and standard pharmaceutical practice.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

Additionally, standard pharmaceutical methods may be employed to control the duration of action. These are well known in the art and include control release preparations and may include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation may be adjusted in order to control release. Additionally, the agent may be incorporated into particles of polymeric materials such as polyesters, polyamino acids, hydrogels, poly (lactic acid) or ethylenevinylacetate copolymers. In addition to being incorporated, these agents may also be used to trap the compound in microcapsules.

Accordingly, the pharmaceutical composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route may be used for administration, a particular route may provide a more immediate and more effective reaction than another route. Local or systemic delivery may be accomplished by administration comprising application or instillation of the formulation into body cavities, inhalation or insufflation of an aerosol, or by parenteral introduction, comprising intramuscular, intravenous, peritoneal, subcutaneous, intradermal, oral, as well as topical administration.

Routes of administration of any of the compositions of the invention include oral, nasal, rectal, intravaginal, parenteral, buccal, sublingual or topical.

The regimen of administration may affect what constitutes an effective amount. The therapeutic formulations may be administered to the subject either prior to or after the onset of a retroviral infection. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat a retroviral infection in the subject. An effective amount of the therapeutic compound necessary to achieve a therapeutic effect may vary according to factors such as the state of the disease or disorder in the subject; the age, sex, and weight of the subject; and the ability of the therapeutic compound to treat a retroviral infection in the subject. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound useful within the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level will depend upon a variety of factors, including the activity of the particular compound employed, the time of administration, the rate of excretion of the compound, the duration of the treatment, other drugs, compounds or materials used in combination with the compound, the age, sex, weight, condition, general health and prior medical history of the subject being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds useful within the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on the unique characteristics of the therapeutic compound and the particular therapeutic effect to be achieved, and the limitations inherent in the art of compounding/formulating such a therapeutic compound for the treatment of a retroviral infection in a subject.

In one embodiment, the compositions of the invention are formulated using one or more pharmaceutically acceptable excipients or carriers. In one embodiment, the pharmaceutical compositions of the invention comprise a therapeutically effective amount of a compound useful within the invention and a pharmaceutically acceptable carrier.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin. In one embodiment, the pharmaceutically acceptable carrier is not DMSO alone.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from individual to individual depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds useful within the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound useful within the invention is from about 1 mg and about 2,500 mg. In some embodiments, a dose of a compound useful within the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., an HIV antiviral) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments therebetween.

In one embodiment, the present invention is directed to a packaged pharmaceutical composition comprising a container holding a therapeutically effective amount of a compound useful within the invention, alone or in combination with a second pharmaceutical agent; and instructions for using the compound to treat, prevent, or reduce one or more symptoms of a retroviral infection in a subject.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the invention, and a further layer providing for the immediate release of a medication for retroviral infection. Using a wax/pH-sensitive polymer mix, a gastric insoluble composition may be obtained in which the active ingredient is entrapped, ensuring its delayed release.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents. For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a retroviral infection in a subject.

The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration:

For oral administration, the compounds useful within the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADR™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration:

For parenteral administration, the compounds useful within the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms:

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 2003/0147952, 2003/0104062, 2003/0104053, 2003/0044466, 2003/0039688, and 2002/0051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention will depend on the age, sex and weight of the subject, the current medical condition of the subject and the nature of the retroviral infection being treated. The skilled artisan will be able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every clay, every other day, every 2 days, every 3 days, every 4 days, or every 5 days.

The active ingredients of the present invention may be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

One or more suitable unit dosage forms having the compositions of the invention, which, as discussed elsewhere herein, may optionally be formulated for sustained release (for example using microencapsulation, see WO 94/07529, and U.S. Pat. No. 4,962,091 the disclosures of which are incorporated by reference herein), may be administered by a variety of routes including parenteral, including by intravenous and intramuscular routes, as well as by direct injection into the diseased tissue. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Extensive preliminary studies revealed that in wild type C57BL/6 mice, the adaptive immune response to influenza virus is primarily mediated by CD8+ cytotoxic T cells and during the first ten days of infection mice lose progressively about 25-30% of their initial body weight. Histopathologic evaluation of the infected lungs, indicate that during the course of the viral infection there is a massive infiltration of lung air space with lymphocytes, polymorphonuclear leucocytes and monocytes. Experimental infection of immunocompetent mice with sub-lethal doses of influenza virus resolves in 10-15 days and is followed by a long lasting immunological memory.

In contrast to wild-type C57BL/6 mice, the results presented herein demonstrate that mice having a genetic deletion of the leukocyte-specific phosphoinositide 3 kinase (PI3K) isoform p110 delta (p110δ, same genetic background as wild-type mice) manifested significantly reduced morbidity after influenza virus infection. At day 6 post infection the numbers of lung CD8+ T cells, NK cells, granulocytes and macrophages were reduced in p110δ deficient mice compared to C57Bl/6 control mice. The number of activated T cells and NK cells were reduced by 3-fold in p110δ deficient mice compared to C57Bl/6 control mice. At the peak of the anti-viral immune response (day 10), the total numbers of lymphocytes, virus-specific CD8+ T cells, B cells, CD4+ T cells, macrophages and granulocytes infiltrating the lungs of p110δ deficient mice were reduced compared to wild-type mice. In addition, the lung viral loads were reduced at days 6 and 10 after infection p110δ deficient mice compared to wild type animals. Without wishing to be bound by any particular theory, it is believed that p110δ deficient mice constitute a valuable mouse model to study the contribution of the immune response induced by influenza virus infection to morbidity symptoms and lung pathology. Therefore, experiments were designed and conducted to determine whether deficient signaling through p110δ induces changes of the immune response to influenza virus that results in decreased morbidity and lung pathology and whether deficient signaling through p110δ inhibits influenza virus infection.

### Example 1

### P110δ Signaling is Required for Influenza Virus Infection/Replication

The results demonstrate that that the p110δ catalytic isoform of the PI3K signaling pathway plays an important role in influenza virus replication. Identifying the PI3K isoforms involved in influenza virus replication is critical as PI3K isoforms regulate many essential homeostatic functions in cells and therefore, non-specific inhibition of these pathways may have considerable toxicity (Crabbe et al., 2007 Trends Biochem Sci 32: 450-456). Deletion of p110 and p110β is embryonic lethal in mice, while deletion of p110γ affects glucose metabolism (Vanhaesebroeck et al., 2005 Trends Biochem Sci 30: 194-204) and cardiac function (Ban et al., 2008 Circ Res 103: 643-653). p110δ□ deficient mice are healthy indicating that toxicity associated with blocking of this isoform would be minimal. The results presented herein demonstrate that p110δ plays a critical role in influenza virus infection.

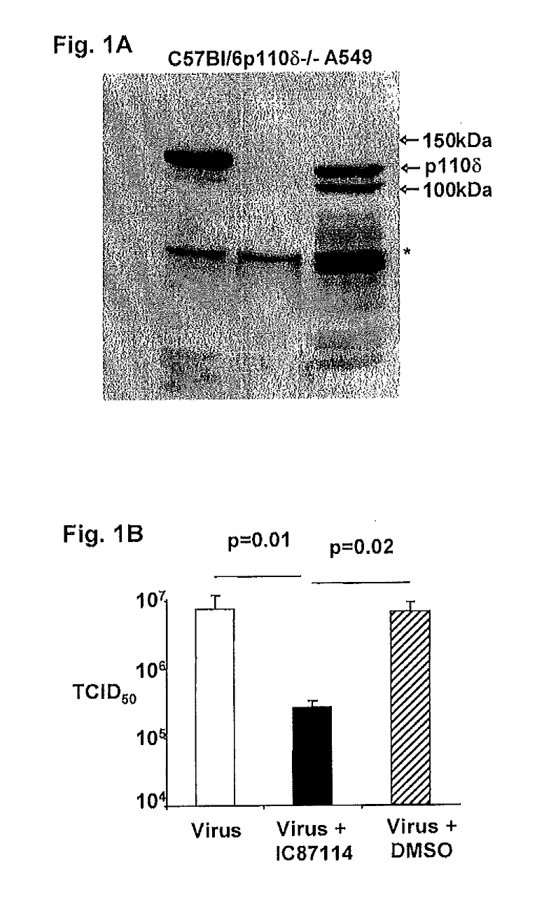

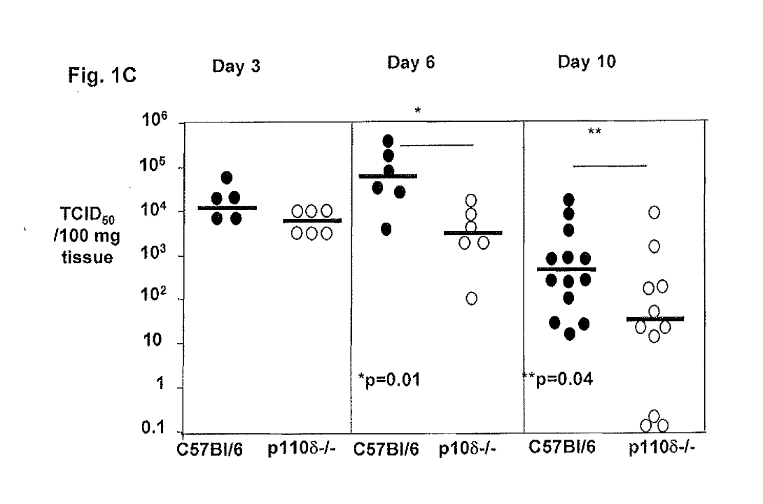

Although p110δ was first described in cells of hematopoietic origin (Vanhaesebroeck et al., 1997, Proc. Natl. Acad, Sci. USA 94:4330-4335; Chantry et al., 1997, J. Biol. Chem. 272:19236-19241), there is evidence that some non-haematopoietic cells also express p110δ (Sawyer et al., 2003 Cancer Res. 63:1667-1675; Eickholt et al., 2007, PLoS ONE 2:e869). The results presented herein demonstrate that lung epithelial cells express p110δ protein by Western blotting (FIG. 1A). Furthermore, it was observed that p110δ is directly involved in influenza virus replication in lung epithelial cells. When the A549 epithelial cell line was infected with influenza virus and treated with IC87114, a selective inhibitor of the p110δ PI3K (Sadhu et al., 2003, J. Immunol. 170:2647-2654), it was observed that viral mRNA production was reduced ˜25-fold in cells compared to untreated cells (FIG. 1B). IC87114 did not affect the survival of A549 cell (data not shown). Without wishing to be bound by any particular theory, it is believed that the reduced virus produced by a human epithelial cell line when p110δ is pharmacologically inhibited suggest that p110δ signaling is directly involved in influenza virus replication and may serve as a target for in vivo infection.

### Example 2

### Examine Viral Loads, Morbidity, Lung Pathology Lung Inflammation and the Level of Pro-Inflammatory Cytokines in the Lungs of p110δ Deficient Mice Compared to C57BL/6 Mice During Influenza Virus Infection

Mice deficient in p110δ and Wild-Type C57Bl/6 controls were infected with influenza virus strain PR8 (3 TCID₅₀). Lungs from the animals were harvested at days 3, 5 and 7 after infection. Flow-cytometry was used to examine the immune cell populations that infiltrated the lungs of p110δ deficient mice and wild-type controls at days 3, 5 and 7 after infection with influenza virus in order to determine whether the lower morbidity of p110δ deficient mice correlated with a reduced cellular infiltration of the lungs. Flow-cytometry can also be used to determine how early after infection can the differences between p110δ deficient mice and wild-type controls be detected.

Experiments were designed to examine whether the morbidity observed in C57BL/6 mice after influenza virus infection correlates with the production of one or more pro-inflammatory cytokines. The mice deficient in p110δ and the wild-type C57Bl/6 controls that were infected with influenza virus discussed elsewhere herein can be used in this study in the following way: a piece of the lung harvested at days 3, 5 and 7 after infection can be used to measure the amount of different pro-inflammatory cytokines (IL-1, IL-6, IL-8, TNFα) by RT-PCR, using commercially available primer pairs.

The role of p110δ PI3K in influenza virus replication was further verified by testing p110δ PI3K deficient mice (p110δ−/− mice). The results presented herein demonstrate that p110δ PI3K was critical to disease pathogenesis and contributed to both morbidity and mortality. It was observed that influenza virus infected p110δ−/− mice had significantly reduced lung viral loads (FIG. 1C).

During influenza virus infection, the air space of the lung is invaded by immune cells that kill the virus-infected epithelial cells and can also secrete inflammatory cytokines. Intense production of proinflammatory cytokines and reduced gas exchange contribute to the morbidity symptoms displayed by influenza virus infected mice (weight loss, labored breathing, lack of appetite, reduced activity). Therefore, lung tissue destruction during viral infection in p110δ deficient mice and in C57Bl/6 control mice, at days 3, 5, 7 and after infection can be evaluated. A piece of the lung collected at the desired time points can be rinsed in PBS, inflated and stored in 4% paraformaldehyde solution before paraffin embedding and processing for histopathologic evaluation.

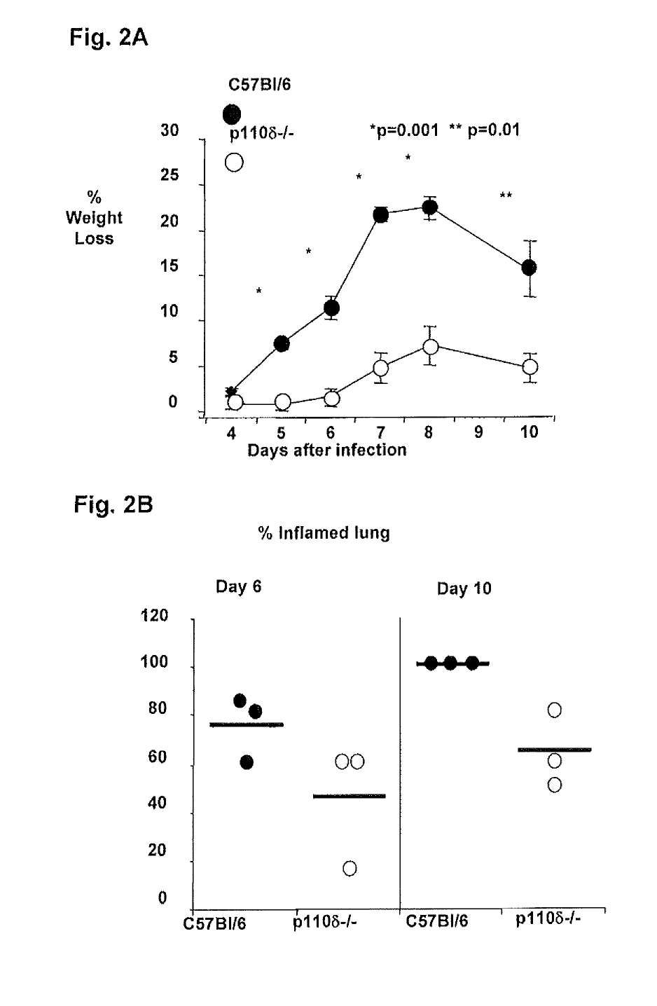

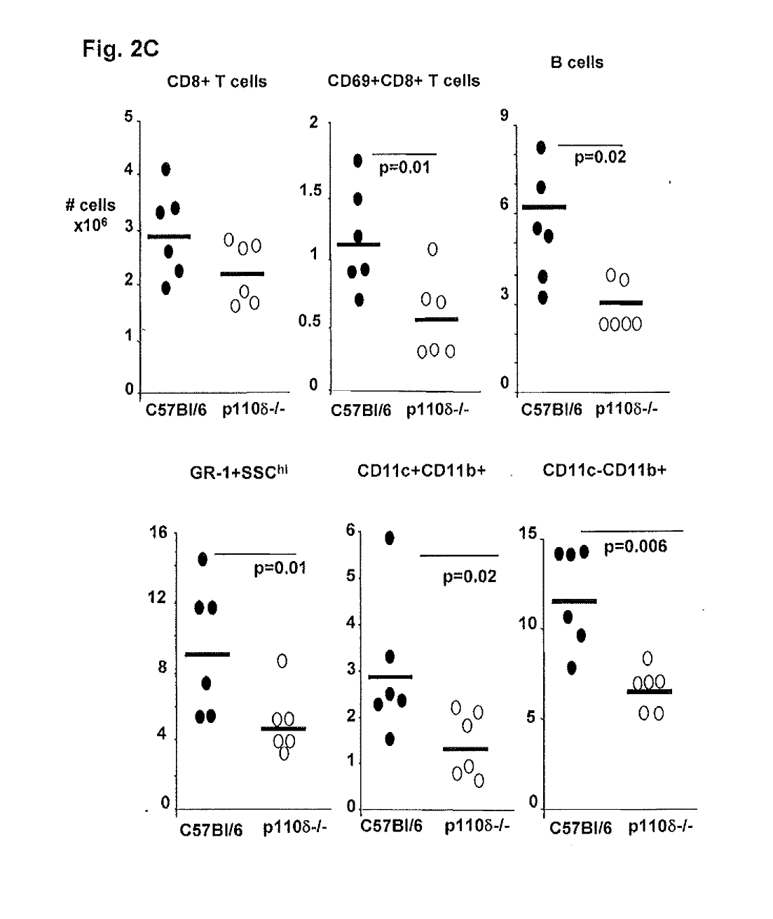

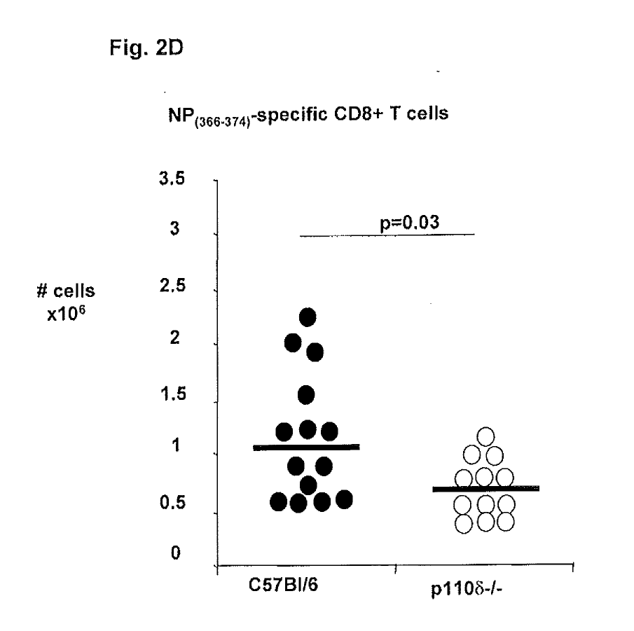

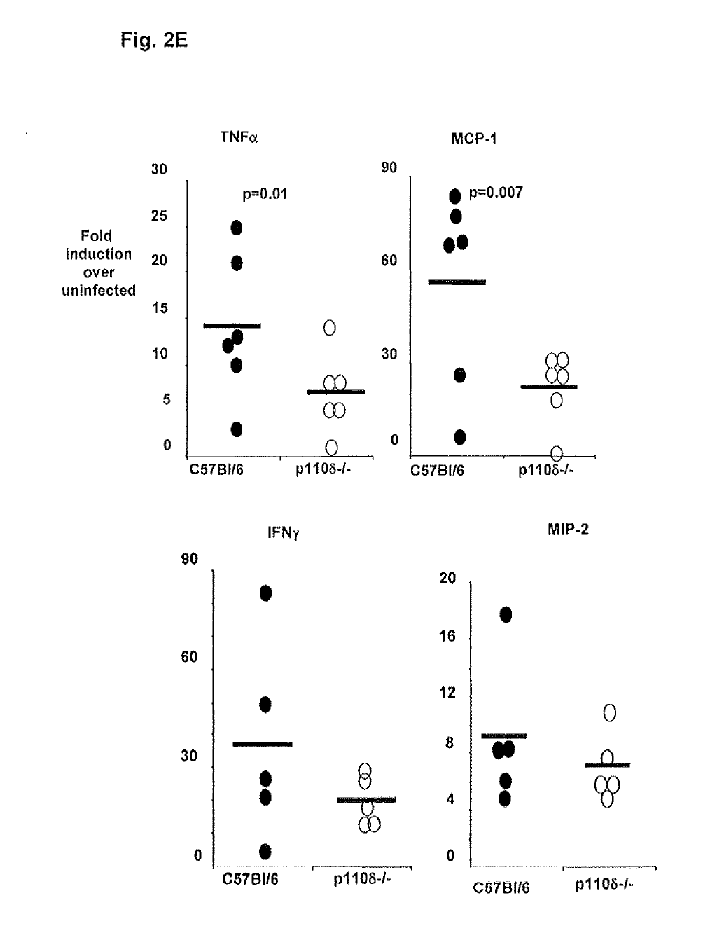

P110δ−/− mice demonstrated reduced weight loss (FIG. 2A) and lung pathology, with p110δ−/− mice presented fewer areas of cellular infiltration in the lung compared to control mice (FIG. 2B). The numbers of inflammatory cells infiltrating the lungs of p110δ−/− mice were also decreased compared to wild type animals (FIG. 2C). At day 6 post infection, granulocytes, macrophages, dendritic cells, activated CD8+ T cells and B cells were reduced in lungs of influenza virus infected p110δ−/− mice (FIG. 2C). At day 10, the peak of the CD8+ T cell response against influenza virus, the immunodominant NP₃₆₆₋₃₇₄-specific CD8+ T cell response was reduced in p110δ−/− mice (FIG. 2D). In addition, the production of inflammatory cytokines believed to contribute to influenza virus morbidity (Hayden et al., 1998 J Clin Invest 101: 643-649; Skoner et al., 1999 J Infect Dis 180: 10-14; Cheung et al., 2002 Lancet 360: 1831-1837; de Jong et al., 2006 Nat Med 12: 1203-1207; Kobasa et al., 2007 Nature 445: 319-323), was also significantly reduced in p110δ−/− mice (FIG. 2E). These findings demonstrate that p110δ PI3K plays an important role in influenza viral replication and pathogenesis.

### Example 3

### Determine Whether Morbidity Associated with Influenza Virus Infection is Reduced By Treating Mice with a Specific Inhibitor of p110δ

A specific inhibitor of p110δ, belonging to the quinazolin family, has been reported (Sadhu et al., 2003 Journal of Immunology 170: 2647-54). C57Bl/6 mice lose up to 30% of their initial body weight during influenza virus infection. The optimal route of administration of this drug can be determined by treating C57Bl/6 mice, either intranasally or intraperitoneally, at day 0 of infection. Also, the optimal dose of inhibitor for each route of administration can be determined. Once these optimal parameters are established, infected C57Bl/6 mice can be treated with the p110δ inhibitor at different time points after infection in order to determine whether it can stop morbidity after viral replication in the lung had started.

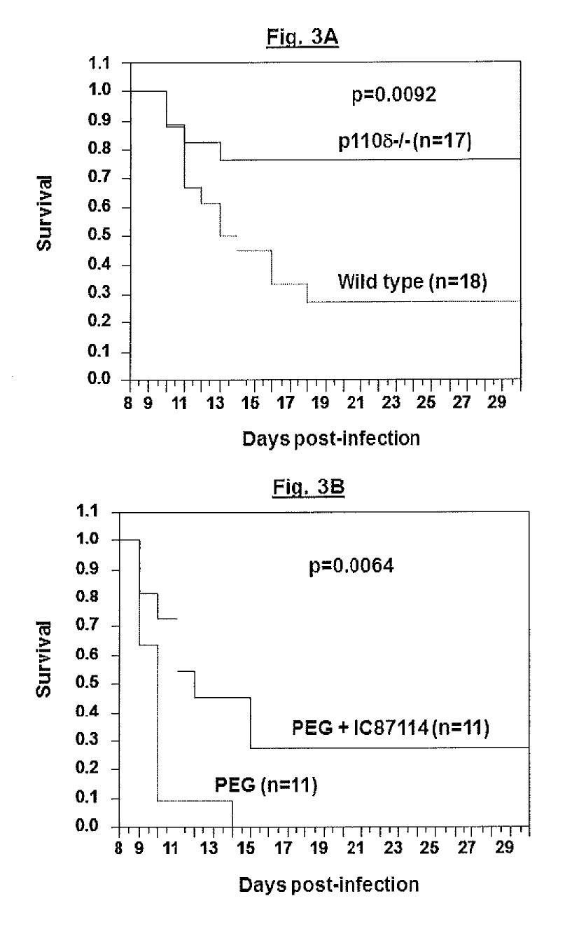

To determine whether inhibition of p110δ signaling could protect against lethal influenza virus infection, lethal challenges (10×LD₅₀) in mice with the virulent for mice influenza virus H7N7 London strain (A/Equine/London/1416/73) was performed (Kawaoka et al., 1991 J Virol 65: 3891-3894). Both p110δ−/− mice and wild type mice treated with IC87114 inhibitor were tested (FIGS. 3A and 3B). It was observed that with both p110δ deficiency and pharmacological inhibition of p110δ led to increased survival after lethal challenge (FIGS. 3A and 3B). The results presented herein demonstrate that P110δ PI3K is an important therapeutic target for influenza virus infection. These findings also provide proof of concept that pharmacological inhibition of p110δ is a useful strategy against severe influenza virus infection.

PI3K isoforms regulate many essential homeostatic functions in cells and therefore inhibiting these pathways may have considerable toxicity (Crabbe et al., 2007 Trends Biochem Sci 32: 450-456). Targeting p110δ to ameliorate pathology and viral replication during influenza virus infection is an attractive strategy as it may not interfere with normal homeostasis of the host. Targeting p10δ in combination with other PI3K isoforms such as p110δγ may synergize and provide additional protective effect.

In summary the results presented herein show that p110δ PI3K plays an important role in the morbidity and mortality of influenza virus infection by controlling viral replication. Therapeutic targeting of such host related molecules may have the advantage of being less prone to the virus developing resistance as mutated virus that does not require p110δ □PI3K would be expected to sustain a cost in replicative fitness and would result in reduced viral replication and morbidity. Pharmacological inhibition of p110δ therefore may present a novel therapeutic strategy for pandemic and seasonal influenza virus infection.

The experiments presented herein also expand the understanding of the contribution of the immune response to lung pathology and morbidity occurring during influenza virus infection. The results presented herein are novel because in the p110δ deficient mice the virus is cleared as efficiently as in wild-type mice, yet these mice did not lose significant weight and their immune response was moderately reduced compared with wild-type controls.

### Example 4

### Inhibition of p110δ Phosphoinositide 3-Kinase Reduces the Magnitude of HIV-1 Infection/Replication without Affecting Mitogenic Activation or the Viability of Human CD4+ T Cells

PBMC were activated with 10 μg/mL PHA-P and 20 U/mL IL-2 for 48 hours and then exposed for 2 hours to the PI3K delta inhibitor IC87114. Cells were subsequently infected with 10⁵ TCID₅₀/ml of HIV-1_{Ba-L} for 1 hour, washed three times, and cultured in 1 mL of fresh media containing 1 μM, 10 μM or 50 μM IC87114 in DMSO or an equivalent concentration of DMSO alone.

Cells were harvested at 2 days post-infection and stained for CD25 and phosphatidyl serine expression in a flow cytometric assay. Viral antigen levels were measured in cell supernatants.

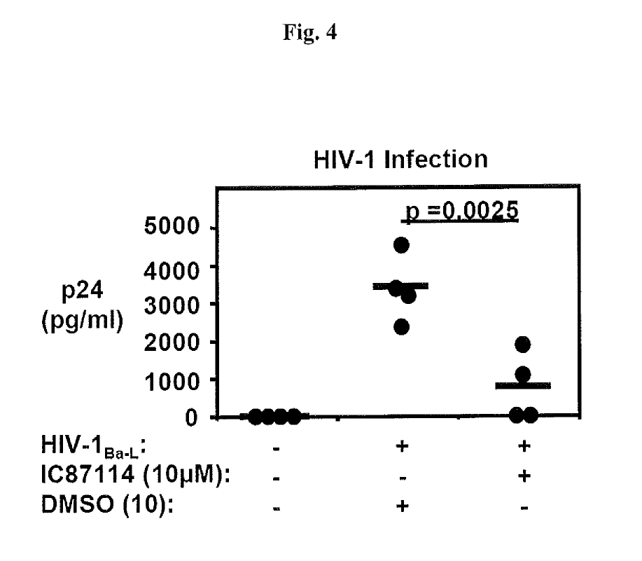

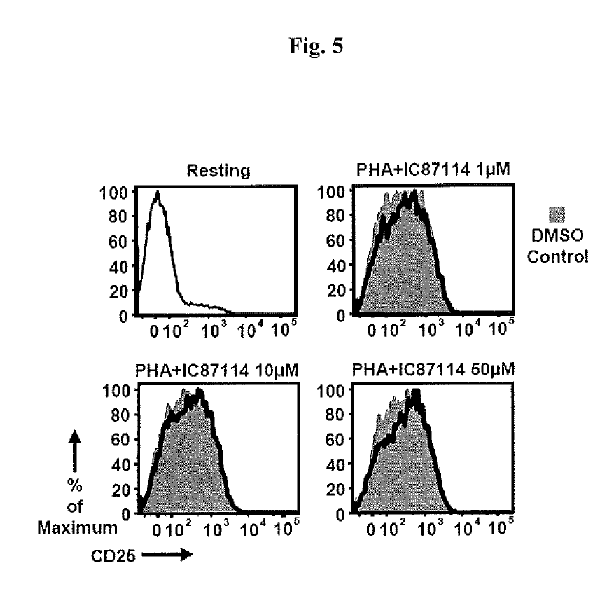

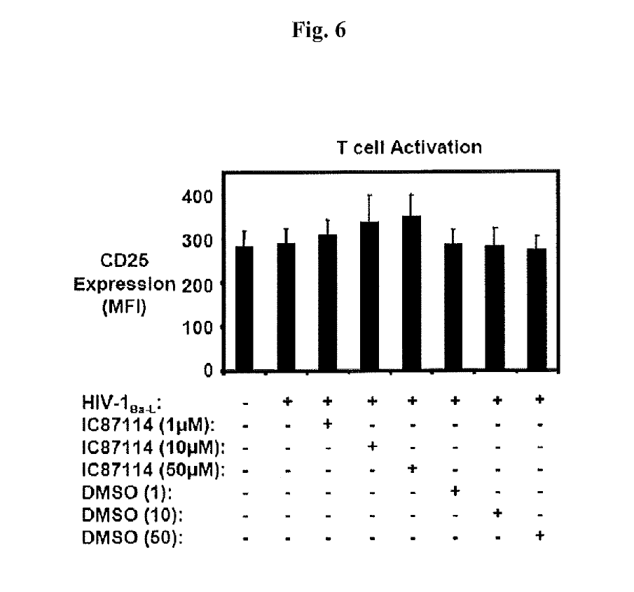

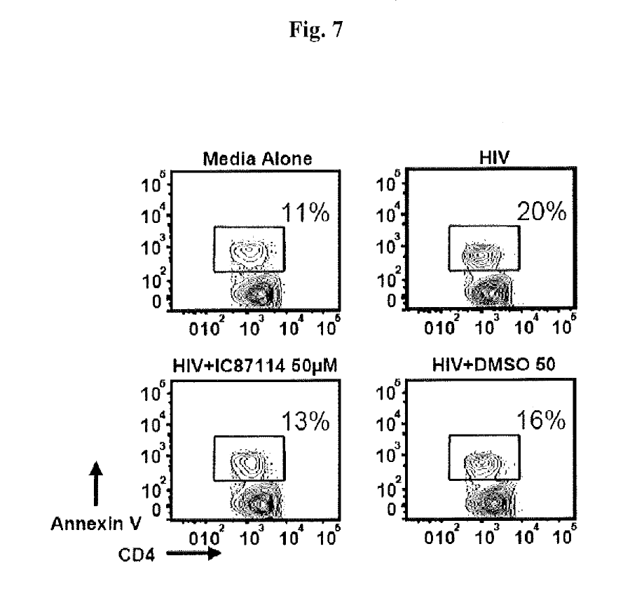

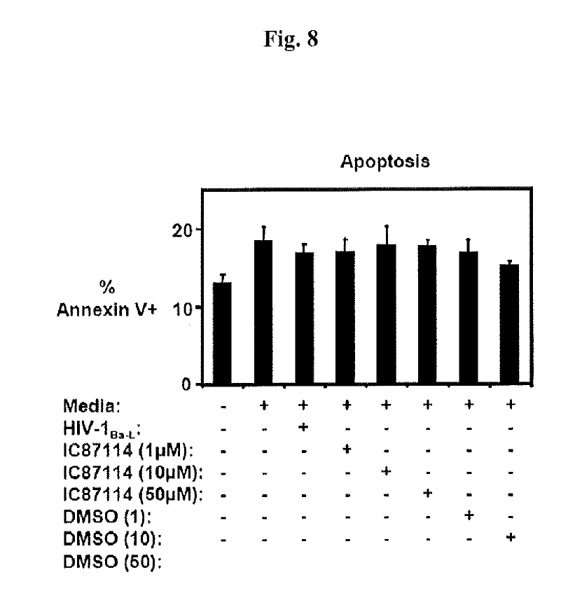

The plot in FIG. 4 illustrates that IC87114 reduced 48 hour HIV-1 p24 levels by approximately 80%, for n=4 donors. The traces in FIG. 5 illustrate the CD25 expression levels on CD4+ T cells exposed to HIV-1 and/or IC87114 from a representative donor. FIG. 6 illustrates that CD25 expression was not affected in PHA-activated CD4+ T cells exposed to HIV-1 and IC87114 at different concentrations, for n=3. FIG. 7 illustrates cell death measured by phosphatidyl serine expression on PHA-activated CD4+ T cells exposed to HIV-1 and IC87114 from a representative donor. FIG. 8 illustrates the frequency of apoptotic CD4+ T cells following exposure to HIV-1 and/or IC87114 at varying concentrations, for n=3.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of inhibiting replication of a retrovirus in a mammalian cell, said method comprising contacting said cell with a pharmaceutically acceptable composition comprising a therapeutically effective amount of an inhibitor of PI3K p110 delta, wherein said contacting inhibits PI3K p110 delta in said cell, thereby inhibiting replication of said retrovirus in said cell.

2. The method of claim 1, wherein said retrovirus is HIV.

3. The method of claim 1, wherein said inhibitor is selected from the group consisting of a small interfering RNA (siRNA), a microRNA, an antisense nucleic acid, a ribozyme, an expression vector encoding a transdominant negative mutant, an antibody, a peptide, a small molecule compound, and combinations thereof.

4. The method of claim 3, wherein said small molecule compound is selected from the group consisting of wortmannin, INK1197, KAR4000, theophylline, CAL-101, CAL-263, Compound (I) (2-[(6-amino-9H-purin-9-yl)methyl]-5-methyl-3-(o-tolyl)-quinazolin-4(3H)one), Compound (II) (2-([(9H-purin-6-yl)thio]methyl)-5-chloro-3-(2-methoxyphenyl)quinazolin-4(3H)-one), Compound (III) (24(4-amino-3-(3-hydroxyphenyl)-1H-pyrazolo[3,4-d]pyrimidin-1-yl)methyl)-5-methyl-3-(o-tolyl)quinazolin-4(3H)-one), Compound (IV) (2-(1-(6-amino-9H-purin-9-yl)ethyl)-3-phenylthieno[3,2-d]pyrimidin-4(3H)-one), Compound (V) (6-(1-((9H-purin-6-yl)thio)ethyl)-3-methyl-5-phenylisoxazolo[5,4-d]pyrimidin-4(5H)-one), Compound (VI) (3-isobutyl-6-methyl-4-oxo-N-(pyridin-3-ylmethyl)-3,4-dihydrofuro[2,3-d]pyrimidine-5-carboxamide), Compound (VII) ((S)-5-chloro-N4-(1-(8-chloro-2-(pyridin-2-yl)quinolin-3-yl)ethyl)pyrimidine-2,4-diamine), Compound (VIII) (4-(2-(1H-indazol-4-yl)-6-((4-(methylsulfonyl)piperazin-1-yl)methyl)thieno[3,2-d]pyrimidin-4-yl)morpholine), Compound (IX) (4-(6-((4-(azetidin-1-yl)piperidin-1-yl)methyl)-2-(6-fluoro-1H-indol-4-yl)thieno[3,2-d]pyrimidin-4-yl)morpholine), Compound (X) (1-((9-ethyl-2-(1H-indol-4-yl)-6-morpholino-9H-purin-8-yl)methyl)-N,N-dimethylpiperidin-4-amine), Compound (XI) (4-(2-(1H-indol-4-yl)-6-((4-methylpiperazin-1-yl)methyl)quinazolin-4-yl)morpholine), Compound (XII) (4-(2-((hexahydropyrrolo[3,4-c]pyrrol-2(1H)-yl)methyl)-5-(1H-indol-4-yl)thiazolo[4,5-d]pyrimidin-7-yl)morpholine), Compound (XIII) (N4(4-(6-fluoro-1H-indol-4-yl)-6-morpholinopyrimidin-2-yl)methyl)nicotinamide), Compound (XIV) (6-(6-fluoro-1H-indol-4-yl)-2-morpholino-N-(2-(pyridin-3-yl)ethyl)pyrimidin-4-amine), Compound (XV) (N-(2-(dimethylamino)ethyl)-4-(6-fluoro-1H-indol-4-yl)-6-morpholinopyrimidine-2-carboxamide), Compound (XVI) (5,5-dimethyl-2-morpholino-5,6-dihydrobenzo[d]thiazol-7(4H)-one), Compound (XVII) ((S)-2-(3-((1H-indol-3-yl)methyl)morpholino)-7,7-dimethyl-5,6,7,8-tetrahydro-4H-thiazolo[5,4-c]azepin-4-one), Compound (XVIII) (6,6-dimethyl-2-(6-phenyl-2H-benzo[b][1,4]oxazin-4(3H)-yl)-6,7-dihydrothiazolo[5,4-c]pyridin-4(5H)-one), Compound (XIX) (5,5-dimethyl-2-(6-phenyl-2H-benzo[b][1,4]oxazin-4(3H)-yl)-5,6-dihydrobenzo[d]-thiazol-7(4H)-one), Compound (XX) (6,6-dimethyl-2-(6-(1-methyl-1H-pyrazol-4-yl)-2H-benzo[b][1,4]oxazin-4(3H)-yl)-6,7-dihydrothiazolo[5,4-c]pyridin-4(5H)-one), Compound (XXI) (2-(6-(1-(2-hydroxy-3-methoxypropyl)-1H-pyrazol-4-yl)-2H-benzo[b][1,4]oxazin-4(3H)-yl)-6,6-dimethyl-6,7-dihydrothiazolo[5,4-c]pyridin-4(5H)-one), Compound (XXII) (6,6-dimethyl-2-(6-(6-methylpyridazin-3-yl)-2H-benzo[b][1,4]oxazin-4(3H)-yl)-6,7-dihydrothiazolo[5,4-c]pyridin-4(5H)-one), Compound (XXIII) (ethyl 2-morpholino-7-oxo-4,5,6,7-tetrahydrobenzo[b]thiophene-3-carboxylate), Compound (XXIV) ((S)-2-(3-((1H-indol-3-yl)methyl)morpholino)-4-methylthiazole-5-carboxamide), Compound (XXV) (2-(6-bromo-2H-benzo[b][1,4]oxazin-4(3H)-yl)-5,5-dimethyl-5,6-dihydrobenzo[d]thiazol-7(4H)-one), Compound (XXVI) ((S)-methyl 3-((4-(6,6-dimethyl-4-oxo-4,5,6,7-tetrahydrothiazolo-[5,4-c]pyridin-2-yl)morpholin-3-yl)methyl)-1-methyl-1H-indole-5-carboxylate), Compound (XXVII) (3,3′-(2,4-diaminopteridine-6,7-diyl)diphenol), Compound (XXVIII) (3-(2,4-diaminopteridin-6-yl)phenol), Compound (XXIX) (6-(1H-indol-4-yl)pteridine-2,4-diamine), Compound (XXX), and a pharmaceutically acceptable salt thereof,
wherein in Compound (XXX) R¹ is N or CH, and R² is a substituent selected from the group consisting of 2,2-(5-thiazolidinyl-2,4-dione)-1-ethylenyl; phenyl; pyridin-2-yl; 1H-indaz-5-yl; 1H-pyrazolo[3,4-b]pyridin-5-yl; 2-amino-3-sulfonamido-pyridin-5-yl; 2-amino-3-[(N-2,4-difluorophenyl)sulfonamido]-pyridin-5-yl; 3-[(N-2,4-difluorophenyl)sulfonamide]-pyridin-5-yl; 3-(2,4-difluorobenzenosulfonylamino)-pyridin-5-yl; and 2-methoxy-3-(2,4-difluorobenzenosulfonylamino)-pyridin-5-yl.

5. The method of claim 1, wherein said composition further comprises at least one anti-HIV drug.

6. The method of claim 5, wherein said at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, protease inhibitors, and combinations thereof.

7. The method of claim 1, wherein said composition further comprises at least one immunomodulator.

8. A method of inhibiting pathogenesis of a retrovirus in a mammalian cell, said method comprising contacting said cell with a pharmaceutically acceptable composition comprising a therapeutically effective amount of an inhibitor of PI3K p110 delta, wherein said contacting inhibits PI3K p110 delta in said cell, thereby inhibiting pathogenesis of said retrovirus in said mammalian cell.

9. The method of claim 8, wherein said retrovirus is HIV.

10. The method of claim 8, wherein said inhibitor is selected from the group consisting of a small interfering RNA (siRNA), a microRNA, an antisense nucleic acid, a ribozyme, an expression vector encoding a transdominant negative mutant, an antibody, a peptide, a small molecule, and combinations thereof.

11. The method of claim 10, wherein said small molecule compound is selected from the group consisting of wortmannin, INK1197, KAR4000, theophylline, CAL-101, CAL-263, Compound (I) (2-[(6-amino-9H-purin-9-yl)methyl]-5-methyl-3-(o-tolyl)quinazolin-4(3H) one), Compound (II) (2-([(9H-purin-6-yl)thio]methyl)-5-chloro-3-(2-methoxyphenyl)quinazolin-4(31-1)-one), Compound (III) (2-((4-amino-3-(3-hydroxyphenyl)-1H-pyrazolo[3,4-d]pyrimidin-1-yl)methyl)-5-methyl-3-(o-tolyl)quinazolin-4(3H)-one), Compound (IV) (2-(1-(6-amino-9H-purin-9-yl)ethyl)-3-phenylthieno[3,2-d]pyrimidin-4(3H)-one), Compound (V) (6-(1-((9H-purin-6-yl)thio)ethyl)-3-methyl-5-phenylisoxazolo[5,4-d]pyrimidin-4(5H)-one), Compound (VI) (3-isobutyl-6-methyl-4-oxo-N-(pyridin-3-ylmethyl)-3,4-dihydrofuro[2,3-d]pyrimidine-5-carboxamide), Compound (VII) ((S)-5-chloro-N4-(1-(8-chloro-2-(pyridin-2-yl)quinolin-3-yl)ethyl)pyrimidine-2,4-diamine), Compound (VIII) (4-(2-(1H-indazol-4-yl)-6-((4-(methylsulfonyl)piperazin-1-yl)methyl)thieno[3,2-d]pyrimidin-4-yl)morpholine), Compound (IX) (4-(6-((4-(azetidin-1-yl)piperidin-1-yl)methyl)-2-(6-fluoro-1H-indol-4-yl)thieno[3,2-d]pyrimidin-4-yl)morpholine), Compound (X) (1-((9-ethyl-2-(1H-indol-4-yl)-6-morpholino-9H-purin-8-yl)methyl)-N,N-dimethylpiperidin-4-amine), Compound (XI) (4-(2-(1H-indol-4-yl)-6-((4-methylpiperazin-1-yl)methyl)quinazolin-4-yl)morpholine), Compound (XII) (4-(2-((hexahydropyrrolo[3,4-c]pyrrol-2(1H)-yl)methyl)-5-(1H-indol-4-yl)thiazolo[4,5-d]pyrimidin-7-yl)morpholine), Compound (XIII) (N-((4-(6-fluoro-1H-indol-4-yl)-6-morpholinopyrimidin-2-yl)methyl)nicotinamide), Compound (XIV) (6-(6-fluoro-1H-indol-4-yl)-2-morpholino-N-(2-(pyridin-3-yl)ethyl)pyrimidin-4-amine), Compound (XV) (N-(2-(dimethylamino)ethyl)-4-(6-fluoro-1H-indol-4-yl)-6-morpholinopyrimidine-2-carboxamide), Compound (XVI) (5,5-dimethyl-2-morpholino-5,6-dihydrobenzo[d]thiazol-7(4H)-one), Compound (XVII) ((S)-2-(3-((1H-indol-3-yl)methyl)morpholino)-7,7-dimethyl-5,6,7,8-tetrahydro-4H-thiazolo[5,4-c]azepin-4-one), Compound (XVIII) (6,6-dimethyl-2-(6-phenyl-2H-benzo[b][1,4]oxazin-4(3H)-yl)-6,7-dihydrothiazolo[5,4-c]pyridin-4(5H)-one), Compound (XIX) (5,5-dimethyl-2-(6-phenyl-2H-benzo[b][1,4]oxazin-4(3H)-yl)-5,6-dihydrobenzo[d]-thiazol-7(4H)-one), Compound (XX) (6,6-dimethyl-2-(6-(1-methyl-1H-pyrazol-4-yl)-2H-benzo[b][1,4]oxazin-4(3H)-yl)-6,7-dihydrothiazolo[5,4-c]pyridin-4(5H)-one), Compound (XXI) (2-(6-(1-(2-hydroxy-3-methoxypropyl)-1H-pyrazol-4-yl)-2H-benzo[b][1,4]oxazin-4(3H)-yl)-6,6-dimethyl-6,7-dihydrothiazolo[5,4-c]pyridin-4(5H)-one), Compound (XXII) (6,6-dimethyl-2-(6-(6-methylpyridazin-3-yl)-2H-benzo[b][1,4]oxazin-4(3H)-yl)-6,7-dihydrothiazolo[5,4-c]pyridin-4(5H)-one), Compound (XXIII) (ethyl 2-morpholino-7-oxo-4,5,6,7-tetrahydrobenzo[b]thiophene-3-carboxylate), Compound (XXIV) ((S)-2-(3-((1H-indol-3-yl)methyl)morpholino)-4-methylthiazole-5-carboxamide), Compound (XXV) (2-(6-bromo-2H-benzo[b][1,4]oxazin-4(3H)-yl)-5,5-dimethyl-5,6-dihydrobenzo[d]thiazol-7(4H)-one), Compound (XXVI) ((S)-methyl 3-((4-(6,6-dimethyl-4-oxo-4,5,6,7-tetrahydrothiazolo-[5,4-c]pyridin-2-yl)morpholin-3-yl)methyl)-1-methyl-1H-indole-5-carboxylate), Compound (XXVII) (3,3′-(2,4-diaminopteridine-6,7-diyl)diphenol), Compound (XXVIII) (3-(2,4-diaminopteridin-6-yl)phenol), Compound (XXIX) (6-(1H-indol-4-yl)pteridine-2,4-diamine), Compound (XXX), and a pharmaceutically acceptable salt thereof, wherein in Compound (XXX) R¹ is N or CH, and R² is a substituent selected from the group consisting of 2,2-(5-thiazolidinyl-2,4-dione)-1-ethylenyl; phenyl; pyridin-2-yl; 1H-indaz-5-yl; 1H-pyrazolo[3,4-b]pyridin-5-yl; 2-amino-3-sulfonamido-pyridin-5-yl; 2-amino-3-[(N-2,4-difluorophenyl)sulfonamido]-pyridin-5-yl; 3-[(N-2,4-difluorophenyl)sulfonamide]-pyridin-5-yl; 3-(2,4-difluorobenzenosulfonylamino)-pyridin-5-yl; and 2-methoxy-3-(2,4-difluorobenzenosulfonylamino)-pyridin-5-yl.

12. The method of claim 8, wherein said composition further comprises at least one anti-HIV drug.

13. The method of claim 12, wherein said at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, protease inhibitors, and combinations thereof.

14. The method of claim 8, wherein said composition further comprises at least one immunomodulator.

15. A method of treating or preventing infection by a retrovirus in a mammal in need thereof, said method comprising administering a pharmaceutically acceptable composition comprising a therapeutically effective amount of an inhibitor of phosphoinositide 3 kinase (PI3K) isoform p110 delta to said mammal, wherein said inhibitor interferes with PI3K p110 delta activation and replication of said retrovirus in said mammal, thereby treating or preventing said infection in said mammal.

16. The method of claim 15, wherein said retrovirus is HIV.

17. The method of claim 15, wherein said inhibitor is selected from the group consisting of a small interfering RNA (siRNA), a microRNA, an antisense nucleic acid, a ribozyme, an expression vector encoding a transdominant negative mutant, an intracellular antibody, a peptide, a small molecule compound, and combinations thereof.

18. The method of claim 17, wherein said small molecule compound is selected from the group consisting of wortmannin, INK1197, KAR4000, theophylline, CAL-101, CAL-263, Compound (I) (2-[(6-amino-9H-purin-9-yl)methyl]-5-methyl-3-(o-tolyl)quinazolin-4(3H) one), Compound (II) (2-([(9H-purin-6-yl)thio]methyl)-5-chloro-3-(2-methoxyphenyl)quinazolin-4(3H)-one), Compound (III) (24(4-amino-3-(3-hydroxyphenyl)-1H-pyrazolo[3,4-d]pyrimidin-1-yl)methyl)-5-methyl-3-(o-tolyl)quinazolin-4(3H)-one), Compound (IV) (2-(1-(6-amino-9H-purin-9-yl)ethyl)-3-phenylthieno[3,2-d]pyrimidin-4(3H)-one), Compound (V) (6-(1-((9H-purin-6-yl)thio)ethyl)-3-methyl-5-phenylisoxazolo[5,4-d]pyrimidin-4(5H)-one), Compound (VI) (3-isobutyl-6-methyl-4-oxo-N-(pyridin-3-ylmethyl)-3,4-dihydrofuro[2,3-d]pyrimidine-5-carboxamide), Compound (VII) ((S)-5-chloro-N4-(1-(8-chloro-2-(pyridin-2-yl)quinolin-3-yl)ethyl)pyrimidine-2,4-diamine), Compound (VIII) (4-(2-(1H-indazol-4-yl)-6-((4-(methylsulfonyl)piperazin-1-yl)methyl)thieno[3,2-d]pyrimidin-4-yl)morpholine), Compound (IX) (4-(6-((4-(azetidin-1-yl)piperidin-1-yl)methyl)-2-(6-fluoro-1H-indol-4-yl)thieno[3,2-d]pyrimidin-4-yl)morpholine), Compound (X) (1-((9-ethyl-2-(1H-indol-4-yl)-6-morpholino-9H-purin-8-yl)methyl)-N,N-dimethylpiperidin-4-amine), Compound (XI) (4-(2-(1H-indol-4-yl)-6-((4-methylpiperazin-1-yl)methyl)quinazolin-4-yl)morpholine), Compound (XII) (4-(2-((hexahydropyrrolo[3,4-c]pyrrol-2(1H)-yl)methyl)-5-(1H-indol-4-yl)thiazolo[4,5-d]pyrimidin-7-yl)morpholine), Compound (XIII) (N-((4-(6-fluoro-1H-indol-4-yl)-6-morpholinopyrimidin-2-yl)methyl) nicotinamide), Compound (XIV) (6-(6-fluoro-1H-indol-4-yl)-2-morpholino-N-(2-(pyridin-3-yl)ethyl)pyrimidin-4-amine), Compound (XV) (N-(2-(dimethylamino)ethyl)-4-(6-fluoro-1H-indol-4-yl)-6-morpholinopyrimidine-2-carboxamide), Compound (XVI) (5,5-dimethyl-2-morpholino-5,6-dihydrobenzo[d]thiazol-7(4H)-one), Compound (XVII) ((S)-2-(3-((1H-indol-3-yl)methyl)morpholino)-7,7-dimethyl-5,6,7,8-tetrahydro-4H-thiazolo[5,4-c]azepin-4-one), Compound (XVIII) (6,6-dimethyl-2-(6-phenyl-2H-benzo[b][1,4]oxazin-4(3H)-yl)-6,7-dihydrothiazolo[5,4-c]pyridin-4(5H)-one), Compound (XIX) (5,5-dimethyl-2-(6-phenyl-2H-benzo[b][1,4]oxazin-4(3H)-yl)-5,6-dihydrobenzo[d]-thiazol-7(4H)-one), Compound (XX) (6,6-dimethyl-2-(6-(1-methyl-1H-pyrazol-4-yl)-2H-benzo[b][1,4]oxazin-4(3H)-yl)-6,7-dihydrothiazolo[5,4-c]pyridin-4(5H)-one), Compound (XXI) (2-(6-(1-(2-hydroxy-3-methoxypropyl)-1H-pyrazol-4-yl)-2H-benzo[b][1,4]oxazin-4(3H)-yl)-6,6-dimethyl-6,7-dihydrothiazolo[5,4-c]pyridin-4(5H)-one), Compound (XXII) (6,6-dimethyl-2-(6-(6-methylpyridazin-3-yl)-2H-benzo[b][1,4]oxazin-4(3H)-yl)-6,7-dihydrothiazolo[5,4-c]pyridin-4(5H)-one), Compound (XXIII) (ethyl 2-morpholino-7-oxo-4,5,6,7-tetrahydrobenzo[b]thiophene-3-carboxylate), Compound (XXIV) ((S)-2-(3-((1H-indol-3-yl)methyl)morpholino)-4-methylthiazole-5-carboxamide), Compound (XXV) (2-(6-bromo-2H-benzo[b][1,4]oxazin-4(311)-yl)-5,5-dimethyl-5,6-dihydrobenzo[d]thiazol-7(4H)-one), Compound (XXVI) ((S)-methyl 3-((4-(6,6-dimethyl-4-oxo-4,5,6,7-tetrahydrothiazolo-[5,4-c]pyridin-2-yl)morpholin-3-yl)methyl)-1-methyl-1H-indole-5-carboxylate), Compound (XXVII) (3,3′-(2,4-diaminopteridine-6,7-diyl)diphenol), Compound (XXVIII) (3-(2,4-diaminopteridin-6-yl)phenol), Compound (XXIX) (6-(1H-indol-4-yl)pteridine-2,4-diamine), Compound (XXX), and a pharmaceutically acceptable salt thereof,
wherein in Compound (XXX) R¹ is N or CH, and R² is a substituent selected from the group consisting of 2,2-(5-thiazolidinyl-2,4-dione)-1-ethylenyl; phenyl; pyridin-2-yl; 1H-indaz-5-yl; 1H-pyrazolo[3,4-b]pyridin-5-yl; 2-amino-3-sulfonamido-pyridin-5-yl; 2-amino-3-[(N-2,4-difluorophenyl)sulfonamido]-pyridin-5-yl; 3-[(N-2,4-difluorophenyl)sulfonamide]-pyridin-5-yl; 3-(2,4-difluorobenzenosulfonylamino)-pyridin-5-yl; and 2-methoxy-3-(2,4-difluorobenzenosulfonylamino)-pyridin-5-yl.

19. The method of claim 15, wherein said composition further comprises at least one anti-HIV drug.

20. The method of claim 19, wherein said at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, protease inhibitors, and combinations thereof.

21. The method of claim 15, wherein said composition further comprises at least one immunomodulator.

22. The method of claim 15, wherein said mammal is a human.

23-30. (canceled)
